(19) United States
(12) Patent Application Publication
(10) Pub. No.: US 2010/0151020 A1
(43) Pub. Date: Jun. 17, 2010

(54) DRUG DELIVERY SYSTEM FOR ZERO ORDER, ZERO ORDER BIPHASIC, ASCENDING OR DESCENDING DRUG DELIVERY OF METHYLPHENIDATE

(76) Inventors: Vered Rosenberger, Modiin (IL); Ofer Aqua, Ofra (IL); Anat Bruchim, Jerusalem (IL); Tehila Beiser, Jerusalem (IL)

Correspondence Address: KENYON & KENYON LLP, ONE BROADWAY, NEW YORK, NY, 10004, US

(21) Appl. No.: 12/639,367

(22) Filed: Dec. 16, 2009

Related U.S. Application Data
(60) Provisional application No. 61/201,963, filed on Dec. 16, 2008.

(51) Int. Cl. A61K 9/24; A61K 31/445; A61P 25/00; B05D 1/00; A61P 3/04

(52) U.S. Cl. 424/465; 424/472; 514/317; 427/2.14

(57) ABSTRACT

The invention is directed to a drug delivery device for controlled release of a drug, such as methylphenidate hydrochloride. The drug deliver device has a drug core, having a plug embedded therein, and at least a first coating that at least partially surrounds the core. The plug may be hollow or solid, and swells upon absorption of water, bursting through the first coating. The drag delivery device enables zero-order drug release profiles as well as more complicated release profiles to be obtained.

### RELATED APPLICATIONS

This application claims benefit of U.S. Provisional Patent Application No. 61/201,963, filed Dec. 16, 2008, the contents of which are incorporated herein in its entirety by reference.

### FIELD OF THE INVENTION

The present invention is directed to a drug delivery device for oral administration of a drug. The drug delivery device provides controlled release of the drug. In particular, the invention is directed to a drug delivery device for the controlled release of methylphenidate.

### BACKGROUND OF THE INVENTION

In the world of controlled release drug delivery systems there have been certain axioms upon which much development has been based. One such axiom is that “flatter is better.” That is, the flatter the delivery curve is over a given period of time, the better the system will behave. It is therefore considered desirable to have delivery systems that give essentially a zero order release profile. In drug delivery systems having a zero order release profile, the amount of drug released is not dependent on the amount left within the delivery system, and remains constant over the entire delivery profile.

Tailoring the drug delivery to the needs of the therapy is another axiom of delivery improvement. One can conceive of therapies that need a sudden burst of drug after several hours of constant delivery or a change in the rate of drug delivery after several hours. A swelling hydrogel tablet delivery system or an eroding tablet delivery system, gives drug delivery that tapers off with time. In the eroding system, the surface that provides drug delivery is shrinking with time so the rate falls off proportionally. If the drug is delivered by diffusion through a non-eroding hydrogel the rate falls off as drug depletion changes the force of the chemical gradient. These systems do not offer the opportunity to carefully tailor the drug release rates.

Zero order delivery has been achieved with the “Oros” osmotic pumps as is documented in many patents held by the Alza Company (e.g., U.S. Pat. No. 3,995,631 to Higuchi, T. et, al., and U.S. Pat. No. 3,977,404 to Theeuwes, F.). The “Oros” system is based on osmotic pressure pushing the drug out of an almost microscopic orifice. The zero order profile is achieved due to the constant, small, cross section of the orifice being the rate determining step in the drug release. The “Oros” system has proven itself in several products but it has limitations. It is most useful for soluble drugs, but has limited applicability with insoluble drugs. The technology of manufacture is somewhat complicated with the need of a laser drilled hole in the semi-permeable coating. The drug release through an almost microscopic hole can also lead to several drawbacks. Clogging of the hole may limit drug release and the streaming of a concentrated solution of drug from the delivery system to the intestinal lumen can cause damage to the intestinal wall (see Laidler, P.; Maslin, S. C.; and Gihome, R. W. Pathol Res Pract 1985 180 (1) 74-76). Delays of the start of drug release can be achieved by coating the system (such as with an enteric coating), but the small orifice may be clogged by the coating and give erratic results in opening (if at all). The “Oros” system is best suited for a simple zero order delivery profile. Complicated patterns can be achieved with the “Oros” such as described in U.S. Pat. No. 5,156,850 to Wong, P. S. et al. and in PCT WO 9823263 to Hamel, L. G. et. al., with concomitant complication of the manufacture and of the system, and without solving the drawbacks of the almost microscopic hole.

Zero order delivery profiles have been achieved with clever manipulation of the geometric surface of drug delivery as embodied in the “Geomatrix” delivery systems. (U.S. Pat. Nos. 4,839,177 to Colombo, P. et. al. and 5,422,123 to Conte, U. et. al.). These systems achieve a zero order profile by sandwiching the drug delivery layer between two layers that are impermeable. Only the drug delivery layer is eroded and the cross-section of the eroding layer is constant. Again, here, there are several drawbacks. The manufacture of the system requires special equipment to produce two and three layer tablets. The system does not easily lend itself to changing the rate of delivery during the release profile. The amount of drug available in the tablet is somewhat limited, as only one of the layers is used for drug delivery. The zero order profile may not be followed up to 100 percent of drug release due to tablet breakup once most of the central layer has eroded.

### BRIEF DESCRIPTION OF THE DRAWINGS

FIG. 1 illustrates a side view of a drug delivery device in accordance with the present invention;

FIG. 2 illustrates a side view of a drug delivery device in accordance with the present invention, wherein the cylindrical plug has burst through the impermeable coating;

FIG. 3 illustrates a side view of a drug delivery device in accordance with the present invention, wherein a void has been formed in the drug core;

FIG. 4 illustrates the in vitro release profile of acetaminophen from a drug delivery device made according to Example 1;

FIG. 5 illustrates the in vitro release profile of acetaminophen from a drug delivery device made according to Example 2;

FIG. 6 illustrates the in vitro release profile of acetaminophen from a drug delivery device made according to Example 3;

FIG. 7 illustrates the in vitro release profile of oxybutynin chloride from a drug delivery device made according to Example 4;

FIG. 8 illustrates a comparison of the in vitro release profile of methylphenidate from a drug delivery device made according to Example 5 to that of the commercial product CONCERTA®;

FIG. 9 illustrates the in vitro release profile of tizanidine from a drug delivery device made according to Example 6;

FIGS. 10(a) and 10(b) illustrate the mean plasma methylphenidate concentrations following single dose oral administration of a 36 mg methylphenidate tablet to twelve healthy male Caucasian volunteers in the fed and fasting states;

FIG. 11 illustrates a methylphenidate hydrochloride drug delivery device of the invention; and

FIG. 12 illustrates the methylphenidate hydrochloride drug delivery device of FIG. 12 after the dissolution of the outer drug coating and the swelling of the plug, which bursts through the first coating.

FIG. 13 illustrates the release rate of 36 mg methylphenidate hydrochloride drug delivery device of example 11.

### SUMMARY OF THE INVENTION

The invention is directed to a drug delivery device, comprising a core that has a plug embedded therein, and a first coating that at least partially surrounds the core. The core comprises a drug and excipients. Preferably, the drug is methylphenidate. The first coating surrounding the core is preferably, but not necessarily, essentially impermeable to the drug. Preferably, the plug is cylindrical. The plug is embedded in the core, and may be hollow (i.e., tubular) or solid. Optionally, the plug contains a drug that may be the same or different from the drug in the core.

The present invention provides a methylphenidate controlled release drug delivery device, comprising: a core, a plug, a coating layer and an outer drug layer, wherein:

a) the core, the plug, and the outer drug layer comprise methylphenidate or a pharmaceutically acceptable salt thereof;

b) the plug is embedded in the core;

c) the coating layer is a delay release layer, essentially impermeable to the drug and at least partially surrounding both the core and the plug;

d) the outer drug layer covers at least a portion of the coating; and

e) the plug, when coated, punctures the coating upon swelling of the plug, thereby forming an orifice in the coating.

The plug expands upon absorbing the little water that permeates the coating, punching a hole in the first coating. The hole that is formed in the coating is the size of the diameter of the solid cylindrical plug or the inner diameter of the hollow cylindrical plug. Thus, the hole is a macroscopic hole. The hole in the coating is filled with either the solid cylindrical plug or the hollow cylindrical plug.

In the case of a solid plug that does not contain a drug, water permeates into the plug, and drug from the core permeates out of the plug. Thus, drug release is very slow up until the point when the plug falls out of the delivery device, as described below. In the case of a hollow plug that does not contain a drug, drug release is effected by entry of water through the macroscopic hole to the core, causing drug dissolution or erosion and the exit of the drug solution or drug suspension through the same hole.

Drug dissolution or erosion is designed to be the rate determining step of drug release, and is constant because of the constant cross section of the hole formed in the coating. In this way, the release of drug from the core occurs at a constant, i.e., zero-order release, rate. The properties of the materials of which the plug is made, i.e., how much axial swelling there is, as well as the geometry of the plug, determine the size of the macroscopic hole and, thus, the rate of the zero order drug release. Changes in the rate of dissolution or erosion of the drug core can also affect the rate of drug release. Thus, it is also possible to obtain non-zero order release profiles, if so desired.

Non-zero order release profiles are easily attainable with the drug delivery device of the invention. Release delays may be obtained by coating the drug delivery device with an outer enteric coating. The enteric coating is applied over the first coating in a smooth fashion. Release delays may also be obtained by varying the thickness of the first coating. A thicker first coating will delay the swelling of the plug, thereby delaying drug release. An immediate release coating of the drug may be applied over either the first coating or the enteric coating. Where an immediate release coating is applied over an enteric coating, an additional drug coating can be applied between the first coating and the enteric coating.

An immediate release layer, as the outer coating of the drug delivery device, preferably provides an immediate release of the drug upon ingestion into the stomach. In contrast, a drug layer between the first layer and the enteric coating will depend on the rate of dissolution of the enteric coating. A drug layer applied between the first layer and an enteric coating will be immediately released upon rapid dissolution of the enteric coating, or will be gradually released by a slowly dissolving enteric coating. Preferably, this occurs in the intestinal tract, resulting in a delayed burst release, followed by a controlled release when the plug bursts through the first coating. Using a first coating that is not completely impermeable to the drug in the core will provide a slow release of drug prior to the plug bursting through the first layer.

The size of the plug, whether the plug contains the drug, and the nature of any excipients used to form the plug, determine the rate of drug delivery from the drug delivery device and whether it provides a descending, ascending, or zero order drug release profile. For example, a descending release profile will be obtained where a hollow plug that does not contain a drug continues to swell after bursting through the first coating, as the diameter of the hole through the hollow plug becomes smaller with time. In contrast, an ascending release profile will be obtained where a hollow plug that does not contain a drug erodes or dissolves after the plug bursts through the first coating, as the diameter of the hole in the plug grows larger with time. In addition, a zero-order release profile will be obtained for hollow plugs that do not contain a drug, where the plug maintains its integrity after bursting through the first coating.

Each of those release profiles may be further modified by providing a plug that comprises the drug.

Abrupt changes in the rate of drug release after a predetermined time can be brought about by having the plug designed to fall out of the core after a certain period. The orifice of drug release will then grow considerably, allowing a more rapid drug release or a burst release to be appended to an extended zero order drug release profile. For example, if the outer diameter of the hollow plug is 6 mm and the inner diameter is 3 mm, then the cross sectional area will grow four fold upon the plug falling out of the system. It is also possible to delay the release of drug by using a solid plug. In which case, drug release is very slow or almost zero until the solid plug falls out of the delivery device.

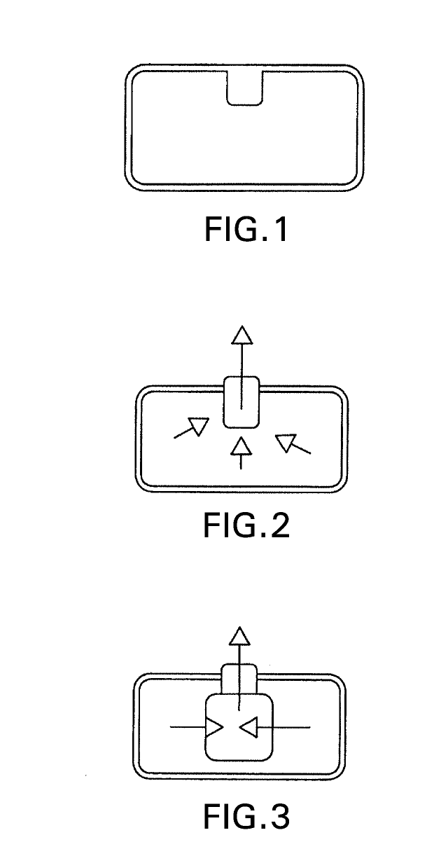

The drug delivery device is preferably made by forming a core comprising a drug, preferably methylphenidate, and excipients, and embedding a plug in the core, as illustrated in FIG. 1. The plug comprises a swellable material, and may optionally further comprise a drug that may be the same as or different from drug in the core. The core is then at least partially coated with the first coating that is optionally essentially impermeable to the drug. The first coating, when impermeable to the drug, serves to prevent any significant drug release from the surface of the drug delivery device. Where the first coating is not completely impermeable to the drug, the rate of diffusion will determine the rate of release until the plug bursts through the first coating, opening a pore.

Small amounts of water do permeate the first coating, causing the plug to swell and burst the surface of the first coating. The result is a partially plugged hole of a defined geometry, as illustrated in FIG. 2. This hole is not a microscopic hole as in the osmotic pump systems. It is a macroscopic hole, having a diameter defined by the inner diameter of the hollow plug or the diameter of the solid plug. Water enters through the hole, and dissolves drug from the core or erodes the core along with drug. The drug flows out of the system through the channel left in the plug. As the drug delivery progresses a void may be formed in the core. This void will be filled with a pool of drug. The rate of drug release from the core is, however, still controlled by the orifice, as illustrated in FIG. 3.

### DETAILED DESCRIPTION OF THE INVENTION

The invention provides a drug delivery device for the controlled release of a drug. The drug delivery device comprises a drug core and a plug embedded in the core. Preferably, the drug is methylphenidate. By the adjustment of the device dimensions (e.g., plug diameter, plug length or height, outer diameter, overall length or height and coating weight gain) the drug delivery device can comprise a variety of dosages of the drug (e.g., 18, 27, 36 and 54 mg).

The present invention provides a methylphenidate controlled release drug delivery device, comprising: a core, a plug, a coating layer and an outer drug layer, wherein:

a) the core, the plug, and the outer drug layer comprise methylphenidate or a pharmaceutically acceptable salt thereof;

b) the plug is embedded in the core;

c) the coating layer is a delay release layer, essentially impermeable to the drug and at least partially surrounding both the core and the plug;

d) the outer drug layer covers at least a portion of the coating; and

e) the plug, when coated, punctures the coating upon swelling of the plug, thereby forming an orifice in the coating.

The core is at least partially coated with a first coating that is optionally essentially impermeable to the drug. Preferably, the first coating has a pH dependant water permeability, e.g., the water permeability at a pH of at least 5 more is greater than the permeability at a pH of less than 5. Preferably, the first coating covers the entire surface area of the core. Optionally, the first coating further covers the plug, for example about 25 percent to about 90 percent, about 40 to about 80 percent, or about 50 percent to about 60 percent of the surface area of the plug. The first coating may cover 100 percent of both the plug and the core. The first coating may also be coated with one or more drug coatings that are the same as or different from the drug in the core. The outer drug layer covers at least a portion of the coating. The drug coating may cover between about 10 percent and about 100 percent of the surface area of the first coating, for example about 25 percent to about 90 percent, about 40 percent to about 80 percent or about 50 percent to about 60 percent.

The core may be a standard pharmaceutical non-expanding core designed to dissolve or erode at a rate that is desired for the therapy at hand. Standard pharmaceutical excipients, such as, fillers, binders, diluents, disintegrants, lubricants, and wetting agents, may be used to form the core. Preferably, the core is in a form of a monolayer core. Useful excipients include, but are not limited to, sucrose (e.g., NUTAB™), Polyethylene glycols (e.g., PEG), microcrystalline cellulose, lactose, sodium lauryl sulfate, polyvinylpyrrolidone, and mixtures thereof. One preferred composition of the core is: 53.9 weight percent sucrose (e.g., NUTAB™), 29 weight percent PEG 8000, 15 weight percent microcrystalline cellulose (e.g., AVICEL pH102™), 1.1 weight percent Acetaminophen, and 1 weight percent magnesium stearate. The diameter of the core preferably ranges from about 7 mm to about 15 mm, with about 9 to about 11 mm being more preferred. The drug content of the core can be from 0.1 to 99 percent by weight of the core, and the drug delivery device can be used to deliver essentially any drug for which oral administration is desired. Preferred drugs include acetaminophen, methylphenidate, oxybutynin, tizanidine, and copaxone.

In a preferred embodiment, a methylphenidate drug delivery device has a plug diameter of about 2 to about 8 mm, and, preferably, about 3 to about 5 mm, a plug length or height of about 1.8 to about 5 mm, preferably, about 2 to about 3 mm, and, more preferably, of about 2.2 to about 2.8 mm, an outer diameter of about 5 to about 11 mm, preferably of about 6 to about 9 mm, an overall length or height of about 4.5 to about 8.5 mm, preferably of about 5.5 to about 7.5 mm, weight gain water permeable coating of about 3 to about 17 mg, preferably of about 4 to about 14 mg, and weight gain immediate released over coat of about 3 to about 60 mg, preferably, about 4 to about 14 mg, more preferably, about 5 to about 60 mg, and, most preferably, about 9 to about 27 mg.

The plug, which is preferably embedded at the surface of the core, may be solid or hollow (i.e., tubular). When the plug is hollow, its outer diameter preferably ranges from about 5 to about 9 mm, with about 7 mm being more preferred. The inner diameter ranges from about 1 mm to about 6 mm, with about 2 mm to about 3 mm being most preferred for an outer diameter of about 7 mm. The plug may comprise a material that further swells after the initial swelling or a material that erodes or dissolves upon contact with fluid after the initial swelling. The plug may be in the form of a bi-layer tablet. One of the layers may be a placebo layer and the other layer may be a drug layer. Alternately, both layers may contain a drug. The drug in the plug may be the same or different as that in the core. Additionally, each layer may contain a different drug.

Preferably, the plug comprises excipients that expand rapidly to break through the first coating while keeping the form and shape of the plug. Preferably, the plug comprises a hydrogel forming agent. Examples of hydrogels that may be used to form the plug include hydroxypropylcellulose, hydroxypropylmethylcellulose, methylcellulose, polyvinylpyrrolidone, polyethylene oxide, and mixtures thereof. Preferably, the plug further comprises at least one of a filler, a binder, a disintegrating agent, an anti-oxidant, and a lubricant. Preferably, the disintegrant comprises at least one superdisintegrant or a combination of at least one disintegrant and at least one superdisintegrant. Examples of disintegrants that may be used include microcrystalline cellulose, methylcellulose, and combinations thereof. Examples of superdisintegrants that may be used include croscarmellose sodium, crospovidone, sodium starch glycolate, and mixtures thereof.

One preferred formulation of the plug is: Hydroxypropylcellulose (e.g., KLUCEL® MF) 35 weight percent, Methylcellulose 1500 34 weight percent, Croscarmellose sodium 30 weight percent, and Magnesium stearate 1 weight percent. Croscarmellose sodium, which is a super disintegrant, serves to cause rapid swelling, while the hydrogel components prevent disintegration of the tablet and keep its geometric integrity.

Other excipients known in the art to posses these properties may be substituted for the preferred formulation as long as they serve to achieve the same function. Any superdisintegrant (e.g., crospovidone, sodium starch glycolate) may be substituted for the croscarmellose and many combinations of hydrogel excipients chosen from the many grades of hydroxypropylcellulose, hydroxpropylmethylcellulose, polyvinylpyrollidone and other polysaccharides may be used. One versed in the art will know how to change the formulation to achieve more or less swelling, or more or less dissolution of the plug during drug release.

The core containing the embedded plug is coated with a first coating that is optionally essentially impermeable to the drug. The first coating is preferably plasticized to a level that facilitates smooth coating, but leaves the coat sufficiently rigid so that it bursts neatly. Examples of plasticizers that may be used include triethylcitrate and polyethylene glycols. The grade of polymer and the amount of plasticizer can be determined by one skilled in the art by routine experimentation. Higher molecular weight polymers will need more plasticizer to keep them in the useful range of rigidity/plasticity. Typically, the plasticizer is present in an amount of from about 5 to about 40 percent by weight of the polymer. A preferred coating comprises ethylcellulose having viscosity of 7 cps (viscosity values for 2 percent (w/v) in aqueous solutions at 20° C.), plasticized with about 20 percent by weight of triethylcitrate. Another preferred coating comprises ethylcellulose having a viscosity of 7 cps (viscosity values for 2 percent (w/v) in aqueous solutions at 20° C.) plasticized with about 20 percent by weight of polyethylene glycol (PEG) 1000.

In a further embodiment, the first coating may be coated with a drug coating. The drug coating may be one that dissolves quickly to provide an immediate pulse of the drug. Alternately, the drug coating may be one that erodes to provide a sustained release of the drug. The drug in the drug coating may be the same or different from the drug in the core. An example of a drug coating that dissolves quickly is one comprising a cationic copolymer based on dimethylaminoethyl methacrylate and neutral methacrylic acid, for example: Poly (butyl methacrylate, (2-dimethyl aminoethyl) methacrylate, methyl methacrylate) 1:2:1, which is commercially available as EUDRAGIT® E 100 granules, and dissolves readily in the stomach.

Not wishing to be bound by any particular theory, it is believed that the use of an aqueous solution of methylphenidate for the outer drug coating provides improved stability of the methylphenidate. Further, it is believed that the use of ethanol for the outer drug coating of the present invention compromises the integrity of the coating layer. Instead, spraying an aqueous solution of the methylphenidate onto the coating layer stabilizes the rate of dissolution over time.

An outer drug layer can be applied to the coating from a solution comprising at least one coating ingredient and a solvent. Preferably, the outer drug layer is sprayed onto the coating. The outer drug layer comprises at least one of a coating polymer and a plasticizer. A drug coating that dissolves quickly is preferably one based on an aqueous solution of purified water or a mixture of water and ethanol, comprising a coating polymer or combination of polymers selected from the list comprising hydroxyproylmethylcellulose, such as METHOCEL™ ES or METHOCEL™ K3 (Dow Chemicals Co), or hydroxy propyl cellulose, such as HPC SSL or HPC SL, preferably having viscosities in the range of 2-8 cps (viscosity values for 2 percent (w/v) in aqueous solutions at 20° C.). The coating may also include plasticizers such as polyethylene glycol, preferably polyethylene glycol having an average molecular weight of about 6,000. Preferably, the drug layer is based on an aqueous solution of purified water or a mixture of water and ethanol, for example a mixture of water and ethanol in ratio of about 95 weight percent to about 5 weight percent, about 80 weight percent to about 20 weight percent, about 60 weight percent to about 40 weight percent and about 50 weight percent to about 50 weight percent. Preferably the solvent contains ethanol in an amount of 50 weight percent or less. Preferably, the drug layer does not contain ethanol. More preferably, the drug layer is based on an aqueous solution of about 100 weight percent purified water.

An example of an eroding drug coating is one that comprises various grades of polyvinylpyrrolidone, hydroxypropyl cellulose, or hydroxypropylmethylcellulose, optionally including one or more plasticizers, as known in the art.

In another embodiment the plug can contain a dose of the same or a different drug as that in the core. This dose can be designed to be delivered slowly from the plug by diffusion or erosion. This delivery device can also be further coated with a drug coating as described above.

In another embodiment, the solid cylinder plug is a bilayer tablet, where one layer expands upon absorbing moisture and bursts through the first coating, while the second layer releases a drug, which can be the same or different as that in the core. The drug may be released in a delayed fashion, the delay being the time needed for the bi-layer tablet to burst the first coating, or the drug may be released in a sustained fashion. This delivery device can also be further coated with a drug coating as described above.

In another embodiment, methylphenidate is incorporated into the core. The core comprises about: 1-10 weight percent drug; 1-20 weight percent microcrystalline cellulose; 60-90 weight percent sucrose; and 0.2-2 weight percent of a lubricant, such as magnesium stearate. The core has a solid plug embedded therein. The solid plug is in the form of a bi-layer tablet. One of the layers comprises about: 1-30 weight percent methylphenidate; 60-95 weight percent lactose; 0-5 weight percent microcrystalline cellulose; and 0.2-2 weight percent lubricant. The other layer, which is the expanding layer that bursts through the first coating, comprises about: 20-50 weight percent hydroxypropylcellulose HF, 20-50 weight percent methylcellulose 1500, 25-40 weight percent croscarmellose sodium, and 0.2-1 weight percent lubricant. The plug is pressed into the surface of the core and the ensemble is coated with a first coating. The first coating comprises about 5-10 mg per tablet of ethylcellulose (e.g., ETHOCEL™ 7 cps) plasticized with about 20 weight percent triethylcitrate. The first coating is then overcoated with a coat comprising EUDRAGIT® E and 0-50 weight percent methylphenidate. Thus, this drug delivery device provides three different doses of methylphenidate. The first dose is an immediate release dose from the outermost (EUDRAGIT® E) coat. The second dose is a short controlled release dose (one to two hours in duration). The third dose is an extended release dose from the core after the solid plug has fallen out. This dose lasts for about 8-12 hours.

In another embodiment, the core comprises about: 7 weight percent methylphenidate; 10 weight percent microcrystalline cellulose; 82 weight percent sucrose; and 1 weight percent magnesium stearate. The plug is a bi-layer tablet, wherein one of the layers is a drug layer and the other layer is an expanding layer. The drug layer weighs about 35 mg and comprises about: 24 weight percent methylphenidate; 70 weight percent lactose; 5 weight percent microcrystalline cellulose; and 1 weight percent magnesium stearate. The expanding layer weighs about 45 mg and comprises about: 35 weight percent hydroxypropylcellulose HF, 34 weight percent methylcellulose 1500; 30 weight percent croscarmellose sodium; and 1 weight percent magnesium stearate. The plug, which has a diameter of about 5 mm, is pressed into the surface of the core and the ensemble is coated with a first coating comprising about 8 mg per tablet of ethylcellulose (e.g., ETHOCEL™ 7 cps (viscosity values for 2 percent (w/v) in aqueous solutions at 20° C.)) plasticized with about 20 weight percent triethylcitrate. The first coating is then over coated with about 18 mg of a drug layer comprising about: 67 weight percent EUDRAGIT® E and 33 weight percent methylphenidate. The entire drug delivery device is an 8 mm tablet weighing about 425 mg.

In yet another embodiment, the core can be a bilayer tablet, wherein each layer contains the same or different drug. Alternately, the upper layer can be a placebo layer to provide either a delay before drug delivery (in the case of the placebo layer) or sequential delivery of two different drugs with independent release profiles or two different release profiles of the same drug.

In another embodiment, a drug may be incorporated into the lower layer of the core, while the other layer comprises a slowly eroding placebo formulation. The drug layer provides a delayed dose of the drug and may be a slow release zero order formulation or may be of short duration slow release so that it approximates a drug burst. The plug is a solid plug comprising a placebo formulation. A first dose of drug is provided by coating the first coating with a drug containing overcoat.

In another embodiment, the core comprises two layers. The lower layer comprises about 2 to about 36 mg tizanidine. This layer may be formulated to release the drug in a sustained or immediate fashion. The upper layer comprises excipients that are eroded slowly over several hours. For example, the upper layer may comprise sucrose, polyvinylpyrrolidone K-30, lactose, and similar excipients.

In a further embodiment, the core has two layers: the upper layer weighs about 210 mg and comprises about 89 weight percent sucrose, about 10 weight percent polyvinylpyrrolidone, and about 1 weight percent magnesium stearate; and the lower layer contains tizanidine and excipients. The solid plug is about 5 mm in diameter and weighs about 50 mg. The plug comprises about 37 weight percent hydroxypropylcellulose HF, about 34 weight percent methylcellulose 1500, about 28 weight percent croscarmellose sodium and about 1 weight percent magnesium stearate. The solid plug is pressed into the upper layer of the core and the entire ensemble is coated first with an impermeable coat of ethylcellulose and then with an overcoat comprising EUDRAGIT® E and about 2 to 8 mg tizanidine. The drug overcoat dissolves readily in gastric fluid giving an immediate burst of tizanidine. The second dose of tizanidine is delayed several hours before it is delivered.

In a preferred embodiment, the drug delivery device of the invention comprises a methylphenidate hydrochloride formulation, comprising a monolayer plug, a drug core, a first coating, and a second drug coating over the first coating. The monolayer plug preferably comprises about 0.1 to about 35 percent, more preferably, about 10 to about 20 percent, most preferably about 17.5 percent methylphenidate hydrochloride, about 0 to about 45 percent, more preferably, about 15 to about 30 percent, most preferably about 22.1 percent filler (e.g., lactose, anhydrous NF), about 0 to about 10 percent, more preferably, about 0.1 to about 5 percent, most preferably about 0.30 percent binder (e.g., polyvinylpyrrolidone), about 0 to about 20 percent, more preferably, about 2 to about 8 percent, most preferably about 5.0 percent of a first disintegrating agent (e.g., microcrystalline cellulose), about 0 to about 35 percent, more preferably, about 5 to about 20 percent, most preferably about 17.1 percent of a second disintegrating agent (e.g., methyl cellulose), about 1 to about 35 percent, more preferably, about 15 to about 27 percent, most preferably about 19.70 percent disintegrating agent (e.g., sodium croscarmellose), about 0 to about 40 percent, more preferably, about 12 to about 24 percent, most preferably about 17.55 percent hydrogel forming agent (e.g., hydroxypropylcellulose), and about 0.1 to about 2 percent, more preferably, about 0.5 to about 1.0 percent, most preferably about 0.75 percent lubricant (e.g., magnesium stearate).

The core preferably comprises about 1 to about 15 percent, more preferably, about 5 to about 10 percent, most preferably about 7.30 percent methylphenidate hydrochloride, about 0 to about 20 percent, more preferably, about 8 to about 15 percent, most preferably about 11.40 percent binder (e.g., microcrystalline cellulose), about 10 to about 80 percent, more preferably, about 30 to about 50 percent, most preferably about 40.15 percent of a first filler (e.g., compressible sugar), about 0 to about 70 percent, more preferably, about 30 to about 50 percent, most preferably about 40.15 percent of a second filler (e.g., a mixture of 75 percent by weight cellulose and 25 percent by weight lactose), and about 0.1 to about 1.5 percent, more preferably, about 0.5 to about 1.2 percent, most preferably about 1.0 percent lubricant (e.g., magnesium stearate).

In a preferred embodiment, the first coating preferably comprises ethylcellulose, an anionic methacrylate copolymer, and a plasticizer.

The first coating preferably comprises about 20 to about 92 percent, more preferably, about 30 to about 60 percent, most preferably about 41.65 percent of a coating polymer (e.g., ethylcellulose) based on the total weight of the coating layer, about 0 to about 70 percent, about 30 to about 70 percent, more preferably, about 30 to about 55 percent, most preferably about 41.65 percent of an enteric coating ingredient, which, preferably, is a polymer (e.g., anionic copolymer based on methacrylic acid and methyl methacrylate, such as poly(methacrylic acid, methyl methacrylate) 1:1, commercially available as EUDRAGIT® L 100 powder) based on the total weight of the coating layer. The coating layer further comprises at least one plasticizer. When present, the plasticizer (e.g., tri-ethylcitrate, tri-ethylcitrate NF) is in an amount of about 5 to about 25 percent, more preferably, about 8 to about 20 percent, and most preferably about 16.70 percent based on the total weight of the coating layer.

The drug coating comprises about 15 to about 60 percent, more preferably, about 25 to about 45 percent, most preferably about 33.3 percent methylphenidate hydrochloride, about 40 to about 85 percent, more preferably, about 50 to about 75 percent, most preferably and about 66.70 percent EUDRAGIT® E-100, as an aminoalkyl methacrylate copolymer coating polymer.

A further preferred embodiment comprises a methylphenidate hydrochloride formulation, comprising a bilayer plug, a methylphenidate hydrochloride core, a first coating, and a drug coating over the first coating. The bilayer plug preferably comprises a hydrogel layer and a drug layer. The hydrogel layer preferably comprises about 0 to about 45 percent, more preferably, about 10 to about 40 percent, most preferably about 34.20 percent of a first disintegrating agent (e.g., methyl cellulose), about 1 to about 50 percent, more preferably, about 25 to about 35 percent, most preferably about 30.20 percent of a second disintegrating agent (e.g., sodium croscarmellose), about 0 to about 50 percent, more preferably, about 22 to about 40 percent, most preferably about 35.10 percent hydrogel forming agent (e.g., hydroxypropylcellulose), and about 0.2 to about 1.2 percent, more preferably, about 0.3 to about 0.75 percent, most preferably, about 0.50 percent lubricant (e.g., magnesium stearate). The drug layer of the bilayer plug preferably comprises about 0.1 to about 35 percent, more preferably, about 20 to about 28 percent, most preferably, about 23.51 percent methylphenidate hydrochloride, about 10 to about 80 percent, more preferably, about 60 to about 75 percent, most preferably, about 70.58 percent filler (e.g., lactose, anhydrous NF), about 0.1 to about 5 percent, more preferably, about 0.5 to about 2.0 percent, most preferably, about 0.71 percent binder (e.g., polyvinylpyrrolidone), about 0 to about 20 percent, more preferably, about 3 to about 8 percent, most preferably about 4.20 percent disintegrating agent (e.g., microcrystalline cellulose), and about 0.1 to about 2 percent, more preferably about 0.5 to about 1.2 percent, most preferably, about 1.0 percent lubricant (e.g., magnesium stearate).

The core preferably comprises about 1 to about 15 percent, more preferably, about 5 to about 10 percent, most preferably about 7.30 percent methylphenidate hydrochloride, about 0 to about 20 percent, more preferably, about 8 to about 15 percent, most preferably about 11.40 percent binder (e.g., microcrystalline cellulose), about 10 to about 80 percent, more preferably, about 30 to about 50 percent, most preferably about 40.15 percent of a first filler (e.g., compressible sugar), about 0 to about 70 percent, more preferably, about 30 to about 50 percent, most preferably about 40.15 percent of second filler (e.g., a mixture of 75 percent by weight cellulose and 25 percent by weight lactose), and about 0.1 to about 1.5 percent, more preferably, about 0.5 to about 1.2 percent, most preferably about 1.0 percent lubricant (e.g., magnesium stearate).

The first coating preferably comprises about 20 to about 92 percent, more preferably, about 30 to about 60 percent, most preferably about 41.65 percent coating polymer (e.g., ethylcellulose), about 0 to about 70 percent, more preferably, about 30 to about 55 percent, most preferably about 41.65 percent anionic methacrylate copolymer coating polymer (e.g., EUDRAGIT® L-100), and about 5 to about 25 percent, more preferably, about 8 to about 20 percent, most preferably about 16.70 percent plasticizer (e.g., triethylcitrate NF).

The drug coating comprises about 15 to about 60 percent, more preferably, about 25 to about 45 percent, most preferably about 33.3 percent methylphenidate hydrochloride, about 40 to about 85 percent, more preferably, about 50 to about 75 percent, most preferably and about 66.70 percent EUDRAGIT® E-100, as an aminoalkyl methacrylate copolymer coating polymer.

A preferred drug delivery device comprising about 36 mg of methylphenidate hydrochloride comprises a monolayer plug, a core, and first and second coatings. The plug comprises about 7.00 mg of methylphenidate hydrochloride, about 8.84 mg of anhydrous lactose, about 0.12 mg of polyvinylpyrrolidone (e.g., POVIDONE K-30), about 2.00 mg of microcrystalline cellulose (e.g., AVICEL PH 102), about 6.84 mg of methylcellulose 1500, about 7.88 mg of croscarmellose (e.g., Ac-di-sol), about 7.02 mg of hydroxypropylcellulose (e.g., KLUCEL® HF), and about 0.30 mg of magnesium stearate.

The core of the 36 mg methylphenidate hydrochloride drug delivery device comprises about 23.00 mg of methylphenidate hydrochloride, about 35.91 mg of microcrystalline cellulose (e.g., AVICEL PH 102), about 126.47 mg of sucrose (e.g., NUTAB™), about 126.47 mg of a 75 percent alpha-lactose monohydrate and 25 percent cellulose powder (e.g., CELLACTOSE 80#), and about 3.15 mg of magnesium stearate.

The first layer of the preferred 36 mg methylphenidate hydrochloride drug delivery device comprises about 3.33 mg of ethylcellulose (e.g., ETHOCEL™ 7 cps), 3.33 mg of anionic copolymers of methacrylic acid and methyl methacrylate, preferably in a ratio of about 1:1, such as EUDRAGIT® L 100, and about 1.34 mg of triethyl-citrate.

The outer drug coating of the preferred 36 mg methylphenidate hydrochloride drug delivery device comprises about 6.00 mg of methylphenidate hydrochloride, and about 12.00 mg of a cationic copolymer, having an average molecular weight of about 150,000 based on dimethylaminoethyl methacrylate and neutral methacrylic esters, such as EUDRAGIT® E-100.

The preferred 36 mg methylphenidate hydrochloride drug delivery device has a plug diameter of about 4.5 mm, a plug length or height of about 2.2 mm, an outer diameter of about 7.8 mm, an overall length or height of about 6.3 mm weight gain water permeable coating of about 7 to about 11 mg, and weight gain immediate released over coat of about 16 to about 20 mg.

A preferred drug delivery device comprising about 18 mg of methylphenidate hydrochloride comprises a monolayer plug, a core, and first and second coatings. The plug comprises about 3.50 mg of methylphenidate hydrochloride, about 4.42 mg of anhydrous lactose, about 0.06 mg of polyvinylpyrrolidone (e.g., POVIDONE K-30), about 1.00 mg of microcrystalline cellulose (e.g., AVICEL PH 102), about 3.42 mg of methylcellulose 1500, about 3.94 mg of croscarmellose (e.g., Ac-di-sol), about 3.51 mg of hydroxypropylcellulose (e.g., KLUCEL® HF), and about 0.15 mg of magnesium stearate.

The core of the 18 mg methylphenidate hydrochloride drug delivery device comprises about 11.5 mg of methylphenidate hydrochloride, about 17.96 mg of microcrystalline cellulose (e.g., AVICEL PH 102), about 63.24 mg of sucrose (e.g., NUTAB™), about 63.24 mg of a 75 percent alpha-lactose monohydrate and 25 percent cellulose powder (e.g., CELLACTOSE 80™), and about 1.58 mg of magnesium stearate.

The first layer of the preferred 18 mg methylphenidate hydrochloride drug delivery device comprises about 1.67 mg of ethylcellulose (e.g., ETHOCEL™ 7 cps), 1.67 mg of anionic copolymers of methacrylic acid and methyl methacrylate, such as poly(methacrylic acid, methyl methacrylate) 1:1, commercially available as EUDRAGIT® L 100, and about 0.67 mg of triethyl citrate.

The outer drug coating of the preferred 18 mg methylphenidate hydrochloride drug delivery device comprises about 3.00 mg of methylphenidate hydrochloride, and about 6.00 mg of a cationic copolymer, having an average molecular weight of about 150,000 based on dimethylaminoethyl methacrylate and neutral methacrylic esters, such as EUDRAGIT® E-100.

The preferred 18 mg methylphenidate hydrochloride drug delivery device has a plug diameter of about 3 mm, a plug length of about 2.2 mm, an outer diameter of about 6 mm, an overall length or height of about 5.5 mm, weight gain water permeable coating of about 3 to about 7 mg, more preferably of about 5.5 mg and weight gain immediate released over coat of about 9 mg.

The preferred 27 mg methylphenidate hydrochloride drug delivery device has a plug diameter of about 3.7 mm, a plug length of about 2.2 to about 2.4 mm, an outer diameter of about 6.3 mm, an overall length or height of about 6.9 mm to about 7.1 mm, weight gain water permeable coating of about 6.5 to about 8 mg, and weight gain immediate released over coat of about 13.5 mg.

The preferred 54 mg methylphenidate hydrochloride drug delivery device has a plug diameter of about 5 mm, a plug length of about 2.6 to about 2.8 mm, an outer diameter of about 9 mm, an overall length or height of about 7.1 mm to about 7.5 mm, weight gain water permeable coating of about 12 to about 16 mg, more preferably of about 13.8 mg and weight gain immediate released over coat of about 27 mg.

In another preferred embodiment, the drug delivery device of the invention comprises a methylphenidate hydrochloride formulation, comprising a plug, preferably a monolayer plug, a drug core, a first coating, and a second drug coating over the first coating. The plug comprises at least one of: a filler comprising anhydrous lactose, a binder comprising polyvinylpyrrolidone, a disintegrating agent comprising at least one of microcrystalline cellulose, methyl cellulose, and sodium croscarmellose, a hydrogel forming agent comprising hydroxypropylcellulose, a anti-oxidant comprising butylated hydroxytoluene, and a lubricant comprising magnesium stearate. The core comprises at least one of a binder, a filler, an anti-oxidant, and a lubricant. When present in the core, the binder comprises microcrystalline cellulose, the filler comprises at least one of compressible sugar and a mixture of alpha-lactose monohhydrate and cellulose powder, the anti-oxidant comprises butylated hydroxytoluene, and the lubricant comprises magnesium stearate. At least one of the core, plug, coating layer, and outer drug layer comprises an antioxidant. Based on the total weight of the monolayer plug, the monolayer plug preferably comprises about 0.1 to about 35 percent, more preferably, about 10 to about 20 percent, about 15 to about 19 percent, most preferably, about 17.5 percent methylphenidate hydrochloride, about 0 to about 45 percent, more preferably, about 15 to about 30 percent, about 20 to about 25 percent, most preferably, about 22.1 percent filler, preferably lactose, preferably, lactose anhydrous NE, about 0 to about 10 percent, more preferably, about 0.1 to about 5 percent, most preferably, about 0.30 percent binder, preferably, polyvinylpyrrolidone, about 0 to about 65 percent first disintegrant, about 0 to about 20 percent, more preferably, about 2 to about 8 percent, most preferably about 5.0 percent microcrystalline cellulose, as a disintegrating agent, about 0 to about 35 percent, more preferably, about 5 to about 20 percent, about 12 to about 20 percent, most preferably about 17.1 percent of a second disintegrating agent (e.g., methyl cellulose), about 1 to about 35 percent, more preferably, about 15 to about 27 percent, about 17 to about 22 percent, most preferably, about 19.70 percent of a third disintegrating agent (e.g., sodium croscarmellose), about 0 to about 40 percent, more preferably, about 12 to about 24 percent, most preferably about 17.5 percent hydrogel forming agent, preferably hydroxypropylcellulose, about 0 to about 0.5 percent, more preferably, about 0.01 to about 0.3 percent, most preferably about 0.1 percent anti-oxidant, preferably, butylated hydroxytoluene and about 0.1 to about 2 percent, more preferably, about 0.5 to about 1.0 percent, most preferably, about 0.7 percent lubricant, which, preferably, is magnesium stearate.

Based on the total weight of the drug delivery device, the plug comprises about 0.1 to about 35, and, preferably, about 1 to about 5 weight percent of methylphenidate hydrochloride, the core comprises 1 to about 15, and, preferably, about 1 to about 10 percent methylphenidate hydrochloride; and the outer drug layer comprises about 0.5 to about 30, and, preferably, about 0.5 to about 5 weight percent methylphenidate hydrochloride.

Based on the weight of the core, the core preferably comprises about 1 to about 15 percent, more preferably, about 5 to about 10 percent, and, most preferably, about 6.8 percent methylphenidate hydrochloride, about 0 to about 20 percent, more preferably, about 8 to about 15 percent, and, most preferably, about 10.7 percent binder, preferably microcrystalline cellulose, about 10 to about 90 percent first filler, about 10 to about 80 percent, more preferably, about 30 to about 50 percent, and, most preferably, about 40.2 percent compressible sugar, as a filler, about 0 to about 70 percent, more preferably, about 30 to about 50 percent, and, most preferably, about 41.4 percent of a second filler (e.g., a mixture of 75 percent by weight alpha-lactose monohydrate and 25 percent by weight cellulose powder), about 0 to about 0.5 percent, more preferably, about 0.01 to about 0.1 percent, most preferably about 0.04 percent anti-oxidant, preferably butylated hydroxytoluene, and about 0.1 to about 1.5 percent, more preferably, about 0.5 to about 1.2 percent, and, most preferably, about 1 percent lubricant, preferably magnesium stearate.

Based on the weight of the first coating, the first coating preferably comprises about 75 to about 95 percent coating polymer, about 20 to about 92 percent, more preferably, about 30 to about 60 percent, about 35 to about 45 percent, and, most preferably, about 41.65 percent coating polymer (e.g., ethylcellulose), about 0 to about 70 percent, preferably about 30 to about 70 percent, more preferably, about 30 to about 55 percent, about 35 to about 45 percent, and, most preferably, about 41.65 percent an anionic methacrylate copolymer coating polymer, preferably EUDRAGIT® L-100, and about 5 to about 25 percent, more preferably, about 8 to about 20 percent, and, most preferably, about 16.70 percent plasticizer, preferably triethylcitrate NF.

Based on the weight of the drug coating, the drug coating comprises about 40 to about 95 percent, more preferably, about 60 to about 90 percent, and, most preferably, about 86.0 percent methylphenidate hydrochloride, about 3 to about 60 percent, more preferably, about 5 to about 30 percent, about 7 to about 15 percent, and, most preferably, and about 10.5 percent coating polymer, preferably hydroxypropylmethyl cellulose of low-viscosity, such as METHOCEL™ E-5, and about 0 to about 10 percent, preferably about 2 to about 10 percent, more preferably, about 2 to about 5 percent, and, most preferably, about 3.5 percent plasticizer, preferably a nonionic polymer having an average molecular weight of about 6,000, such as polyethylene glycol (PEG) 6000.

In a preferred embodiment, the monolayer plug comprises about 2 to about 12 mg of methylphenidate hydrochloride, the core comprises about 8 to about 35 mg methylphenidate hydrochloride; and the outer drug layer comprises about 2 to about 14 mg methylphenidate hydrochloride.

In a preferred embodiment of a drug delivery device comprising about 18 mg of methylphenidate hydrochloride, the plug comprises about 2.5 to about 4.5 mg of methylphenidate hydrochloride, the core comprises about 9.8 to about 11.8 mg methylphenidate hydrochloride; and the drug layer comprises about 2.7 to about 4.7 mg methylphenidate hydrochloride.

In a preferred embodiment of a drug delivery device comprising about 27 mg of methylphenidate hydrochloride, the plug comprises about 4 to about 6 mg of methylphenidate hydrochloride, the core comprises about 15 to about 17 mg methylphenidate hydrochloride; and the drug coating layer comprises about 4 to about 6 mg methylphenidate hydrochloride.

In a preferred embodiment of a drug delivery device comprising about 36 mg of methylphenidate hydrochloride, the plug comprises about 6 to about 8 mg of methylphenidate hydrochloride, the core comprises about 20.5 to about 22.5 mg methylphenidate hydrochloride; and the drug coating layer comprises about 6.5 to about 8.5 mg methylphenidate hydrochloride.

In a preferred embodiment of a drug delivery device comprising about 54 mg of methylphenidate hydrochloride, the plug comprises about 9 to about 11 mg of methylphenidate hydrochloride, the core comprises about 31 to about 33 mg methylphenidate hydrochloride; and the drug coating layer comprises about 10 to about 12 mg methylphenidate hydrochloride.

A further preferred drug delivery device comprising about 36 mg of methylphenidate hydrochloride comprises a monolayer plug, a core, and first and second coatings. The plug comprises about 7.0 mg of methylphenidate hydrochloride, about 8.84 mg of anhydrous lactose, about 0.12 mg of polyvinylpyrrolidone (e.g., POVIDONE K-30), about 2.00 mg of microcrystalline cellulose (e.g., AVICEL PH 102), about 6.84 mg of methylcellulose 1500, about 7.88 mg of croscarmellose (e.g., Ac-di-sol), about 7.00 mg of hydroxypropylcellulose (e.g., KLUCEL® HF), about 0.04 mg of Butylated hydroxytoluene (BHT) and about 0.28 mg of magnesium stearate.

The core of the 36 mg methylphenidate hydrochloride drug delivery device comprises about 21.5 mg of methylphenidate hydrochloride, about 33.61 mg of microcrystalline cellulose (e.g., AVICEL PH 102), about 126.47 mg of sucrose (e.g., Di-pac™), about 130.35 mg of a 75 percent alpha-lactose monohydrate and 25 percent cellulose powder (e.g., CELLACTOSE 80™), about 0.13 mg of Butylated hydroxytoluene (BHT) and about 2.93 mg of magnesium stearate.

The first layer of the preferred 36 mg methylphenidate hydrochloride drug delivery device comprises about 3.33 mg of ethylcellulose (e.g., ETHOCEL™ 7 cps), 3.33 mg of anionic copolymers of methacrylic acid and methyl methacrylate, preferably in a ratio of about 1:1, such as EUDRAGIT® L 100, and about 1.34 mg of triethyl citrate.

The outer drug coating of the preferred 36 mg methylphenidate hydrochloride drug delivery device comprises about 7.5 mg of methylphenidate hydrochloride, about 0.89 mg of Hydroxypropylmethyl cellulose of low-viscosity, such as METHOCEL™ E-5, and about 0.30 mg of a nonionic polymer having an average molecular weight of about 6,000, such as PEG 6000, as a plasticizer.

The more preferred 36 mg methylphenidate hydrochloride drug delivery device has a plug diameter of about 4.5 mm, a plug length or height of about 2.3 mm, an outer diameter of about 7.8 mm, an overall length or height of about 6.5 mm weight gain water permeable coating of about 7 to about 11 mg, and weight gain immediate released over coat of about 6 to about 11 mg.

A preferred drug delivery device comprising about 18 mg of methylphenidate hydrochloride comprises a monolayer plug, a core, and first and second coatings. The plug comprises about 3.50 mg of methylphenidate hydrochloride, about 4.42 mg of anhydrous lactose, about 0.06 mg of polyvinylpyrrolidone (e.g., POVIDONE K-30), about 1.00 mg of microcrystalline cellulose (e.g., AVICEL PH 102), about 3.42 mg of methylcellulose 1500, about 3.94 mg of croscarmellose (e.g., Ac-di-sol), about 3.50 mg of hydroxypropylcellulose (e.g., KLUCEL® HF), about 0.02 mg of Butylated hydroxytoluene (BHT) and about 0.15 mg of magnesium stearate.

The core of the 18 mg methylphenidate hydrochloride drug delivery device comprises about 10.8 mg of methylphenidate hydrochloride, about 16.81 mg of microcrystalline cellulose (e.g., AVICEL PH 102), about 63.24 mg of sucrose (Di-pac™), about 65.17 mg of a 75 percent alpha-lactose monohydrate and 25 percent cellulose powder (e.g., CELLACTOSE 80™), about 0.06 mg of Butylated hydroxytoluene (BHT) and about 1.46 mg of magnesium stearate.

The first layer of the preferred 18 mg methylphenidate hydrochloride drug delivery device comprises about 1.67 mg of ethylcellulose (e.g., ETHOCEL™ 7 cps), 1.67 mg of anionic copolymers of methacrylic acid and methyl methacrylate, preferably in a ratio of about 1:1, such as EUDRAGIT® L 100, and about 0.67 mg of triethyl citrate.

The outer drug coating of the preferred 18 mg methylphenidate hydrochloride drug delivery device comprises about 3.7 mg of methylphenidate hydrochloride, about 0.45 mg of Hydroxypropylmethyl cellulose of low-viscosity, such as METHOCEL™ E-5, and about 0.15 mg of a nonionic polymer having an average molecular weight of about 6,000, such as PEG 6000, as a plasticizer.

Preferably, all the dosages of the drug comprise the same distribution of ingredients in all tablets parts (e.g., plug, core, coating, etc). Therefore, preferably, all the tablets ingredients are weight proportional between the doses.

The more preferred 18 mg methylphenidate hydrochloride drug delivery device has a plug diameter of about 3.2 mm, a plug length of about 2.0 mm, an outer diameter of about 6 mm, an overall length or height of about 5.6 mm, weight gain water permeable coating of about 3 to about 7 mg, more preferably of about 4 mg and weight gain immediate released over coat of about 4 to about 5 mg, more preferably about 4.3 mg.

The preferred 27 mg methylphenidate hydrochloride drug delivery device has a plug diameter of about 3.7 mm, a plug length of about 2.2 to about 2.4 mm, an outer diameter of about 6.3 mm, an overall length or height of about 7.1 mm to about 7.3 mm, weight gain water permeable coating of about 6.5 to about 8 mg, and weight gain immediate released over coat of about 6 to about 7 mg, more preferably about 6.4 mg.

The preferred 54 mg methylphenidate hydrochloride drug delivery device has a plug diameter of about 5 mm, a plug length of about 2.5 to about 2.7 mm, an outer diameter of about 9 mm, an overall length or height of about 7.3 mm to about 7.7 mm, weight gain water permeable coating of about 12 to about 16 mg, more preferably of about 14.0 mg and weight gain immediate released over coat of about 12 to about 13 mg, more preferably about 12.8 mg.

In a preferred embodiment, methylphenidate release profile is 36 percent or less for the first 30 minutes, between about 25 and about 40 percent is released for 1 hour, between about 35 and about 50 percent is released for 2 hours, between about 40 and about 65 percent is released for 4 hours, and 65 percent or above is released for 8 hours when tested in Apparatus 2 (Paddle) at 37° C., 100 RPM in 900 ml of media, when the first two hours were tested in buffer pH=1.2 (70 ml of fuming 37 percent Hydrochloric acid and 20 g sodium chloride up to 10 L, purified water), then additional 10 hours in buffer pH6.8 (9 g sodium hydroxide, 68 g Potassium Phosphate to 10 L purified water).

In another preferred embodiment, the present invention describes a stable methylphenidate drug delivery device. Preferably, the total impurities after one month at 40° C. and 75 percent relative humidity are less than about 0.5 percent, more preferably, less than about 0.3 percent, less than about 0.2 percent, less than about 0.15 percent by weight of methylphenidate content of the final tablet. Preferably, the total impurities does not increase by more than about 100 percent, preferably more than about 50 percent by weight of methylphenidate content of the final tablet. Preferably the total impurities increase by less than about 40 percent, and most preferably less than about 30 percent by weight of methylphenidate content of the final tablet after one month at 40° C. and 75 percent relative humidity.

In another preferred embodiment, the amount of total impurities at production (t=0) is less than about 0.25 percent, preferably less than about 0.2 percent, more preferably less than about 0.15 percent by weight of methylphenidate content of the final tablet.

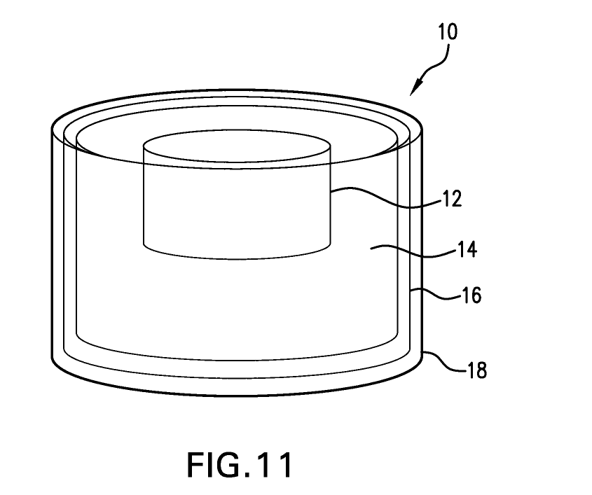

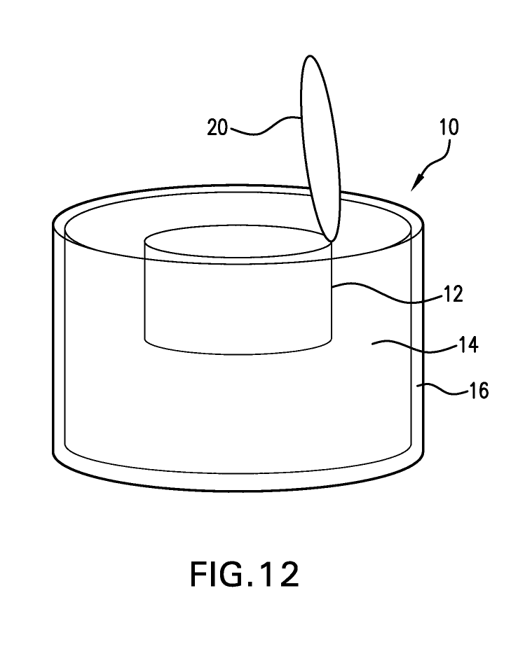

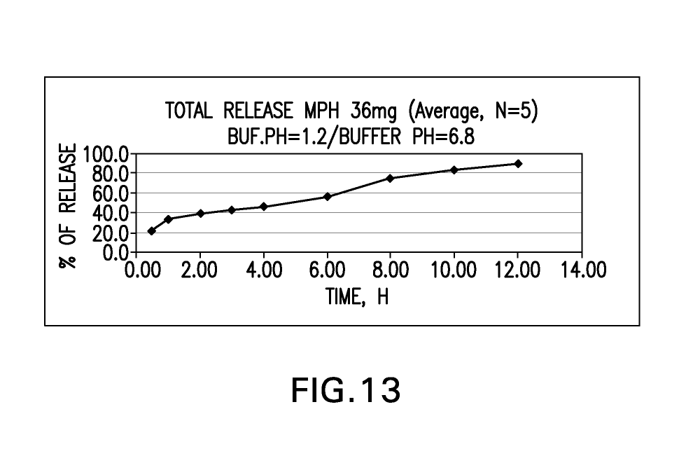

A preferred methylphenidate hydrochloride drug deliver device of the invention is illustrated in FIG. 11. The illustrated methylphenidate hydrochloride drug delivery device comprises a plug 12, a core 14, an at least partially water permeable first coating 16, and an outer coating comprising methylphenidate hydrochloride 18. In an acidic environment, such as the stomach, the outer drug coating 18 dissolves rapidly, providing an immediate release of methylphenidate hydrochloride. Water will also permeate through the first coating 16 into the plug 12 and the core 14. As the plug 12 absorbs water, it expands, and eventually bursts through the first coating 16, as illustrated in FIG. 12. The portion 20 of the first coating 16 produces an opening having the diameter of the plug. As the plug degrades, the methylphenidate hydrochloride is then released through the opening from both the plug 12 and the core 14.

Thus, the drug delivery device is capable of providing various release profiles, including a zero order drug release profile, a biphasic drug release profile, a triphasic drug release profile, an ascending drug release profile, or a descending drug release profile.

The invention further provides a method of making a drug delivery device by forming a core comprising a drug and excipients; embedding a plug in the core; and at least partially coating the core with a first coating that is optionally essentially impermeable to the drug.

The plug may be formed using standard tableting machines with a punch of proper design. It may be formed by direct compression or standard granulation techniques. The plug may comprise two layers. One of the layers may be a placebo layer and the other layer may be a drug layer. Alternately, both layers may contain a drug. The drug in the plug may be the same or different as that in the core. Additionally, each layer may contain a different drug.

The core, with the plug embedded therein, may be produced in a standard press coat tableting machine (e.g., KILLIAN RUD or equivalents). The plug is fed as a preformed plug and the core formulation is fed as a mixture of powders or as a granulate. The press coat tableting machine is operated with the inner tablet off center to place it at the surface of the core. The entire assembly is coated with a first coating such as ethylcellulose or EUDRAGIT® RS.

In one embodiment, the core is a bi-layer tablet where the lower layer is the drug containing layer and the upper layer is a placebo layer. The plug is embedded at the surface of the upper layer. In this embodiment, the core is produced in a press coating tableting machine (KILLIAN RUD) modified to have two powder adding stations before the tablet adding station (so that the tablet is at the surface and not in the center of the tablet as in a “press coated” tablet) and fitted with normal concave punches. The lower layer is formed by blending the drug and excipients and filling the mixture into the die at the first fill station. The upper layer is formed by blending the appropriate excipients and feeding the mixture at the second station. The delay in the release of the drug in the lower layer can be controlled by adding more or less of the mixture which forms the upper placable layer to the second fill station. The plug is then fed as a preformed tablet at the third station using the KILLIAN RUD automatic mechanism for adding preformed tablets to the powder bed.

In another preferred embodiment, the plug and the core are formed by employing compaction as dry granulation of the ingredients, without any necessity of granulation solution. By avoiding the need of granulation solution, the drug delivery may be applicable to a wide rage of drugs, including moisture sensitive drugs. Moreover, the compaction simplify the process since, as opposed to wet granulation, it is a one-step process.

In another embodiment the present invention provides a process for applying (e.g., spraying) a drug layer comprising methylphenidate or a pharmaceutically acceptable salt thereof to a drug delivery device comprising applying the drug layer from a solution comprising methylphenidate or a pharmaceutically acceptable salt thereof, at least one coating ingredient and a solvent selected from the group consisting of water or a mixture of water and ethanol. Preferably, the solvent contains ethanol in an amount of 50 weight percent or less, more preferably, the solvent is purified water. Optionally, the drug layer is applied onto a coating comprising coating ingredients having essentially low aqueous dissolution, preferably polymers which dissolve in ethanol (e.g., ethylcellulose and anionic copolymer based on methacrylic acid and methyl methacrylate).

The methylphenidate drug delivery device according to the present invention may be used for treating conditions mediated by methylphenidate selected from the list comprising attention-deficit hyperactivity disorder, Postural Orthostatic Tachycardia Syndrome, narcolepsy, lethargy, depression, neural insult, and obesity.

Having thus described the invention with reference to certain preferred embodiments, it is further illustrated by the following non-limiting examples.

### Example 1

### Zero Order Release

### Formation of Hollow Plug:

The hollow plug was formed by mixing the excipients in Table 1 in a plastic bag for about 5 minutes. Magnesium Stearate (1 weight percent) was then added and the mixture mixed for a further one minute. The plug was formed in a MANESTY F3 single punch tableting machine using a punch that gives the geometry in Table 2.

TABLE 1

| Material | Weight Percent |
|---|---|
| Hydroxypropylcellulose (KLUCEL ® | 35 |
| MF) | |
| Methylcellulose 1500 | 34 |
| Croscarmellose Sodium | 30 |

TABLE 2

| | |
|---|---|
| Height | 2.9 mm |
| Diameter | 7 mm |
| Inner Diameter | 3.5 mm |

### Formation of Core:

The core was formed by mixing the excipients and drug shown in Table 3 for about five minutes in a plastic bag. Magnesium Stearate (1 weight percent) was then added and the mixture mixed for another minute. The drug delivery device was formed using a MANESTY F3 single punch fitted with a 10 mm diameter normal concave punch by filling with the excipient and active mixture, placing the hollow cylindrical plug on the mixture, and pressing. Drug delivery devices were obtained that had the physical characteristics described in Table 4.

TABLE 3

| Material | Weight Percent |
|---|---|
| Sucrose (NUTAB ™) | 53.9 |
| PEG 8000 | 29 |
| Microcrystalline cellulose (Avicel | 15 |
| pH102) | |
| Acetaminophen | 1 |

TABLE 4

| | |
|---|---|
| Weight | 570 mg |
| Height | 5.6 mm |
| Diameter | 10 mm |
| Hardness | 10.3 kp |

### Coating:

The drug delivery device was coated with a coat of ethylcellulose using the conditions in Table 5:

TABLE 5

| | |
|---|---|
| Ethylcellulose concentration | 3% w/w |
| Triethylcitrate (plasticizer) Concentration | 0.6% w/w |
| Flow rate | 1.6 ml/min |
| Air pressure | 0.5 bar |
| Bed temperature | 36-45° C. |

The ethylcellulose coating was about 14-18 mg/tablet.

### In Vitro Release:

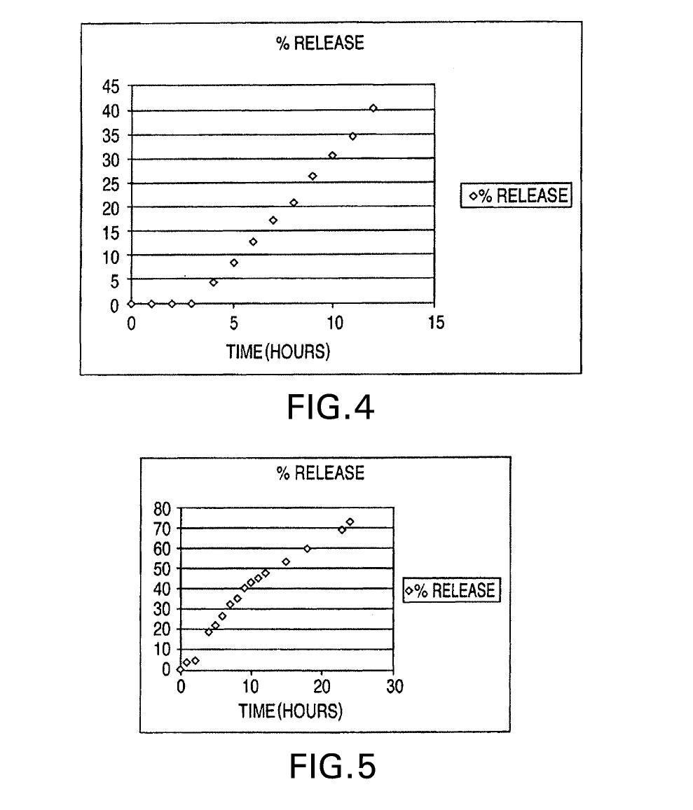

The in vitro release was measured in 900 ml of water in a dissolution bath at 37 degrees and 100 RPM. The amount of acetaminophen released was measured by UV at 243 nm. A clear zero order release profile for 4-12 hours with a release rate of about 5 percent per hour, is obtained. The results are shown in Table 6 and in FIG. 4.

TABLE 6

| Time | % Release |
|---|---|
| 0 | 0 |
| 1 | 0 |
| 2 | 0 |
| 3 | 0 |
| 4 | 4.41 |
| 5 | 8.63 |
| 6 | 12.92 |
| 7 | 17.38 |
| 8 | 20.79 |
| 9 | 26.61 |
| 10 | 30.75 |
| 11 | 34.69 |
| 12 | 40.48 |

### Example 2

### Zero Order Release

### Formation of Hollow Cylindrical Plug:

A hollow cylindrical plug was formed by mixing the excipients shown in Table 7 in a plastic bag for 5 about minutes. Magnesium Stearate (1 weight percent) was then added and the mixture mixed for another minute. The cylindrical plug was pressed in a MANESTY F3 single punch tableting machine using a punch that gives the geometry in Table 8.

TABLE 7

| | Weight |
|---|---|
| Material | Percent |
| Hydroxypropylcellulose (KLUCEL ® HF) | 50.3 |
| Hydroxypropylmethylcellulose | 16.7 |
| (METHOCEL ™ K-15) | |
| Croscarmellose Sodium | 22 |
| Tannic Acid | 10 |

TABLE 8

| | |
|---|---|
| Height | 2.9 mm |
| Diameter | 7 mm |
| Inner Diameter | 3.5 mm |

### Formation of Core: Same as Example 1

### Coating: Same as Example 1

### In Vitro Release: Same as Example 1

### Results

The results of the in vitro release are given in Table 9 and FIG. 5.

TABLE 9

| | % |
|---|---|
| time | Release |
| 0 | 0 |
| 1 | 3.3 |
| 2 | 4.59 |
| 4 | 18.57 |
| 5 | 22.35 |
| 6 | 26.38 |
| 7 | 32.26 |
| 8 | 35.62 |
| 9 | 40.26 |
| 10 | 43.19 |
| 11 | 45.24 |
| 12 | 47.82 |
| 15 | 53.22 |
| 18 | 60.2 |
| 23 | 69.38 |
| 24 | 73.86 |

As shown in Table 9, an essentially zero order drug release pattern over 24 hours with a release rate of about 3 percent per hour is obtained. The hollow cylindrical plug in this example was designed to swell to a larger extent than the one in Example 1 by changing the formulation of the cylindrical plug. The larger swelling leads to earlier drug release (earlier breach of the impermeable ethylcellulose coating) and to a slower release rate. The inner diameter of the cylindrical plug is made smaller by the swelling of the material. The smaller diameter of the channel in the cylindrical plug gives a lower release rate of the soluble drug.

### Example 3

### Biphasic Release

### Formation of Hollow Cylindrical Plug: Same as in Example 1

### Formation of Core:

The core was formed by mixing the excipients and drug shown in Table 10 for five minutes in a plastic bag. Magnesium Stearate (1 weight percent) was then added and the mixture mixed for another minute. The drug delivery device was formed using a MANESTY F3 single punch fitted with a 10 mm diameter normal concave punch by filling with the excipient and active mixture, placing the hollow cylindrical plug on the mixture, and pressing. A drug delivery device was obtained that had the physical characteristics described in Table 11.

TABLE 10

| Material | Weight % |
|---|---|
| Sucrose (Nutab ™) | 40.5 |
| PEG 4000 | 24.6 |
| Sodium Lauryl Sulfate (SLS) | 5.0 |
| POVIDONE K-30 | 5.0 |
| 75% alpha-lactose monohydrate and 25% | 22.8 |
| cellulose (CELLACTOSE 80 ™) | |
| Acetaminophen | 1.1 |

TABLE 11

| | |
|---|---|
| Weight | 535 mg |
| Height | 6.3 mm |
| Diameter | 10 mm |
| Hardness | 7.4 kp |

### Coating: Same as Example 1

### In Vitro Release: Same as Example 1

### Results

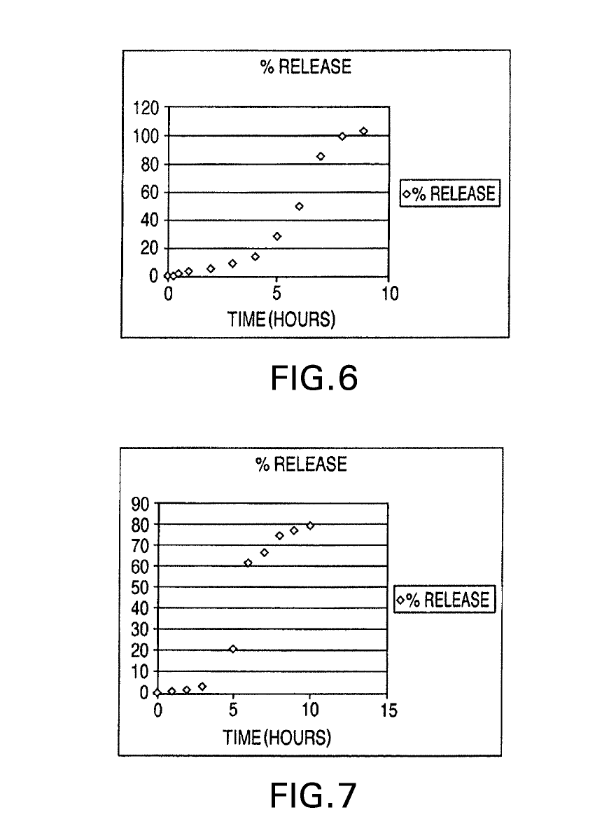

The results of the in vitro release are given in Table 12 and FIG. 6.

TABLE 12

| time | % Release |
|---|---|
| 0 | 0 |
| 0.25 | 0.31 |
| 0.5 | 1.35 |
| 1 | 2.86 |
| 2 | 5.03 |
| 3 | 8.6 |
| 4 | 12.96 |
| 5 | 28.12 |
| 6 | 49.55 |
| 7 | 84.93 |
| 8 | 99.01 |
| 9 | 102.16 |

As shown in Table 12, a zero order release rate similar to that in example 2 for the first four hours followed by an accelerated rate of release is obtained. The hollow cylindrical plug fell out of the tablet after four hours, thus widening the opening for drug release from =3.5 mm (the exact diameter is somewhat different due to the swelling of the formulation of the cylindrical plug) to 7 mm. The formulation of the core, specifically the SLS in the formulation, leads to the cylindrical plug falling out after a predetermined time.

### Example 4

### Biphasic Release

### Formation of Hollow Cylindrical Plug:

### Same as in Example 1

### Formation of Core:

The core was formed by mixing the excipients and drug in Table 13 for five minutes in a plastic bag. Magnesium Stearate (1 weight percent) was then added and the mixture mixed for another minute. The drug delivery device was formed using a MANESTY F3 single punch fitted with a 10 mm diameter normal concave punch by filling with the excipient and active mixture, placing the hollow cylindrical plug on the mixture, and pressing. A drug delivery device was obtained that had the physical characteristics described in Table 14. In this example Oxybutynin chloride was used as an active in place of Acetaminophen.

TABLE 13

| Material | Weight % |
|---|---|
| Sucrose (Nutab ™) | 38.5 |
| PEG 4000 | 30.0 |
| Sodium Lauryl Sulfate (SLS) | 8.0 |
| POVIDONE K-30 | 20.0 |
| Oxybutynin chloride | 2.5 |

TABLE 14

| | |
|---|---|
| Weight | 610 mg |
| Height | 6.4 mm |
| Diameter | 10 mm |
| Hardness | 7.3 kp |

### Coating: Same as Example 1

### In Vitro Release: Same as Example 1

### Results

The results of the in vitro release are given in Table 15 and FIG. 7.

TABLE 15

| | % |
|---|---|
| time | Release |
| 0 | 0 |
| 1 | 1.33 |
| 2 | 2 |
| 3 | 3.67 |
| 5 | 20.83 |
| 6 | 61 |
| 7 | 66.83 |
| 8 | 74.67 |
| 9 | 77.33 |
| 10 | 79.33 |

As shown in Table 15, a slow release the first three hours followed by a rapid burst of drug and then a rapid release phase to the finish of the drug release, is obtained. Again here, SLS in the formulation leads to the cylindrical plug falling out after a predetermined time, providing a more rapid drug release.

### Example 5

### Triphasic Release of Methylphenidate

### The Concept

To obtain triphasic release there needs to be three reservoirs of the drug. The first reservoir must be capable of immediate release while the next two reservoirs are delayed release. The first of the delayed release doses has a relatively short release period while the last, main reservoir, provides an extended release. To achieve this profile the following delivery system was developed, using the invention described herein. The first dose of drug is delivered from an outer overcoat that is readily soluble in gastric fluid. This coat contains about 6 mg of drug. The cylindrical plug that bursts through the impermeable first coating is a solid, bi-layer cylindrical plug. The upper layer is a swelling layer that bursts through the impermeable coating while the lower layer contains another dose of about 6-7 mg of the drug. The core is the main reservoir of drug, containing about 23 mg of the drug. The core is designed to give a zero order extended release through the hole made by the bi-layer embedded solid cylindrical plug. When the system is placed in gastric fluid the overcoat dissolves immediately giving a first dose of immediate release. There is a delay while the embedded cylindrical plug swells and punctures the impermeable coat. The drug dose contained in the lower layer of the embedded cylindrical plug is released over a short period (½ to 2 hours). The drug in the core starts to release. The drug in this reservoir is released over 6-8 hours in a zero order fashion.

### The Cylindrical Plug

The cylindrical plug was a hi-layer 5 mm flat beveled tablet produced using a KILLIAN RUD tablet press. The drug layer was prepared by first granulating methylphenidate with lactose and then blending with microcrystalline cellulose and subsequently magnesium stearate. The granulation was carried out by blending 150 parts lactose (DMV International) with 50 parts methylphenidate (Mallinkrodt Inc.) on a Zanchetta Rotolab machine. Water (20 parts) was added to wet the mass while mixing at 350 rpm and then at 500 rpm. The mass was milled through a 1.6 mm screen (Erweka), dried in a fluidized bed drier (Aeromatic Laboratory Drier) at 40 C to a moisture content of less than 1.5 percent, and milled again through a 0.8 mm screen. This granulate, 94 parts, was blended with 5 parts microcrystalline cellulose (AVICEL™ pH102 FMC International) for several minutes, then one part of magnesium stearate NF/EP (Mallinkrodt Inc.) was added and blended for another minute. The swellable gel layer was formed by blending 35.2 parts hydroxypropylcellulose (KLUCEL® HF, Aqualon Ltd.), 34.2 parts methylcellulose 1500 (Dow Chemical Inc.), and 30.1 parts croscarmellose sodium (AC-DI-SOL, FMC International) for 5 minutes. Magnesium stearate at 0.5 parts was added and the blend mixed for a further minute. The drug containing layer weighed 30 mg while the swelling layer weighed 45 mg. The tablets had a hardness of about 3-6 kP. The drug content of the drug layer was 7 mg.

### The Core

The core was formed by pressing the bi-layer solid cylindrical plug into a blend of methylphenidate and excipients. The blend was formed by mixing 6.6 parts of methylphenidate, 10.0 parts of microcrystalline cellulose, and 82.4 parts compressible sucrose (Nutab™ DMV International) for several minutes, adding 1 part magnesium stearate, and mixing for one minute. The tablets were compressed using a MANESTY F3 single punch machine, fitted with 8 mm flat beveled punches, with manual placement of the preformed bi-layer cylindrical plug in the powder bed. The drug delivery device (the bi-layer cylindrical plug embedded at the surface of the core) had a diameter of 8 mm, a weight of 425 mg and a hardness of 17-20 kP. The drug content of the core was 23 mg.

### The Drug Delivery Device

The drug delivery device was coated with an insoluble coating in an Erweka coating pan heated with an air gun. The first coating solution was 3.0 percent ethylcellulose (ETHOCEL™ 7 cps Dow Chemical Inc.) and 0.6 percent triethyl citrate (Rhom Pharma Ltd.) in ethanol. The solution was sprayed through a 1 mm nozzle using 0.5 bar atomizing air with a solution flow of 1-2 ml/minute. The solution flow was varied to prevent sticking of the drug delivery device and to keep the temperature of the tablet bed between 35° to 40° C. The spraying process was stopped at a weight gain of 7-9 mg per drug delivery device forming the impermeable coat. A drug overcoat was formed on the insoluble coating. The drug overcoat comprised a solution of 2.5 weight percent EUDRAGIT® E100 (Rhom Pharma Ltd.) and 1.25 weight percent methylphenidate dissolved in ethanol. This solution was sprayed onto the ethylcellulose coated drug delivery devices to a weight gain of 18 mg, giving a drug content of 6 mg for this layer.

### Results

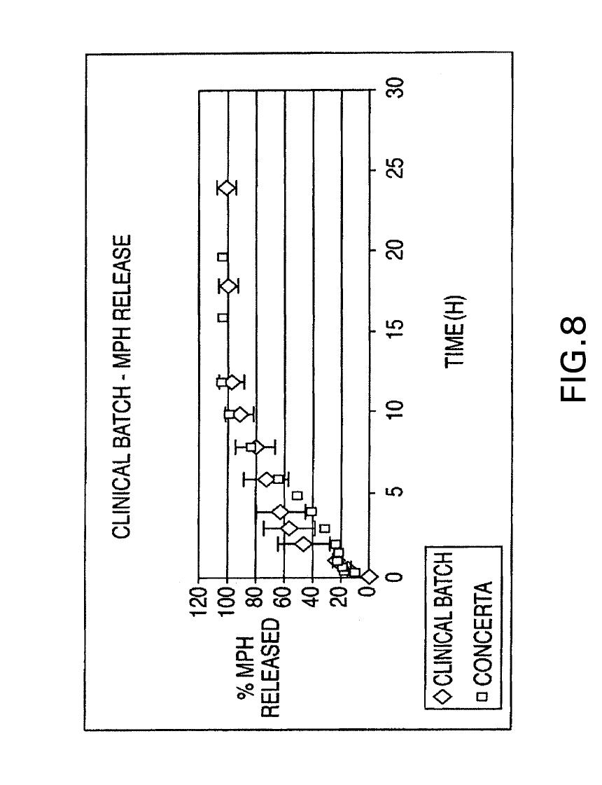

The drug delivery device was tested in a Hanson dissolution bath at 37 C in 900 ml of media. The first two hours were tested in 0.1N HCl. After two hours the drug delivery device was transferred to distilled water. The methylphenidate in the device was determined by an HPLC method on a cyano column using an aqueous buffer (pH 4): acetonitrile system with UV detection at 210 nm. The results are shown in Table 16 and in FIG. 8. FIG. 8 illustrates a comparison of the release profile obtained with the drug delivery device of Example 5 to that of the commercial methylphenidate product CONCERTA®. The profile illustrates an immediate release, followed by a delay and then an extended release profile which has a slower and then a faster portion. The in vitro release profile for the device in Example 5 illustrates the same elements. An initial burst is seen immediately followed by another burst of drug at two hours. This is then followed by an essentially zero order drug release up to 8 to 10 hours.

TABLE 16
Release Profile of methylphenidate in vitro

| time (h) | vessel 1 | vessel 2 | vessel 3 | vessel 4 | vessel 5 | vessel 6 | Average | Stdev |
|---|---|---|---|---|---|---|---|---|
| 0 | 0 | 0 | 0 | 0 | 0 | 0 | 0.0 | 0.0 |
| 0.5 | 11.8 | 18.7 | 18.7 | 13.4 | 15.2 | 19 | 16.1 | 3.1 |
| 1 | 21.9 | 22.4 | 19.3 | 22.8 | 28.3 | 21.6 | 22.7 | 3.0 |
| 2 | 63.8 | 38.7 | 22.4 | 55.4 | 65.1 | 29.9 | 45.9 | 18.1 |
| 3 | 72.5 | 44.5 | 28 | 67.2 | 73.9 | 51 | 56.2 | 18.2 |
| 4 | 76.7 | 50.7 | 32.6 | 72.4 | 77.6 | 63.4 | 62.2 | 17.7 |
| 6 | 83.1 | 73.8 | 41.7 | 79 | 84.4 | 74.1 | 72.7 | 15.8 |
| 8 | 87.1 | 87.8 | 52.3 | 85.5 | 90.2 | 79.8 | 80.5 | 14.2 |
| 10 | 98.3 | 95.6 | 73.1 | 93.5 | 99.8 | 90.4 | 91.8 | 9.7 |
| 12 | 98.9 | 110.3 | 84.2 | 95.2 | 101.1 | 92.9 | 97.1 | 8.7 |
| 18 | 99.7 | 112 | 96 | 95.2 | 101.1 | 92.9 | 99.5 | 6.8 |
| 24 | 104.2 | 112 | 99.5 | 95.2 | 101.1 | 92.9 | 100.8 | 6.8 |

Thus, the drug delivery device of the invention has been shown to be capable of generating complicated delivery patterns in vitro. In the case of Example 5, it delivers two bursts of drug delayed by about 1 hour, followed by a zero order release of the drug up to 8-10 hours.

### Example 6

### Delayed 2nd Dose of Tizanidine

### The Concept

To obtain a delayed second dose of a drug after an immediate release dose, two reservoirs of drug are necessary. The immediate release layer is contained in an outer overcoat as in Example 5. The second dose of drug may be in the cylindrical plug or in the core. Placing the second dose in the core allows more flexibility in designing the length of the delay. To obtain the ability to control the delay the cylindrical plug is a solid swelling plug while the core is a bi-layer tablet. The upper layer is a placebo layer that erodes at a predetermined rate depending on its formulation and the size of the hole punched in the impermeable coat. Alternately, the thickness of the placebo layer can be the determining factor in the delay time. Beneath this layer is the drug layer which releases the drug in a short, controlled release pattern.

### The Cylindrical Plug

The cylindrical plug was formed by pressing a blend of excipients in a MANESTY F3 single punch tableting machine fitted with 5 mm flat faced punches. The blend was formed by mixing 37.1 parts hydroxypropylcellulose (KLUCEL® HF, Aqualon Ltd), 34.5 parts methylcellulose 1500 (Dow Chemical Inc.), and 27.4 parts croscarmellose sodium (AC-DI-SOL, FMC International) for five minutes. 1.0 part of magnesium stearate NF/EP (Mallinkrodt Inc.) was added and the blend mixed for another minute. The cylindrical plug weight was 50 mg and its hardness was 2-6 kP.

### The Core

The core is a bi-layer tablet where the lower layer is the drug containing layer and the upper layer is a placebo layer. The cylindrical plug is embedded at the surface of the upper layer. The core was produced on a KILLIAN RUD tablet machine modified to have two powder adding stations before the tablet adding station (so that the tablet is at the surface and not in the center of the tablet as in a “press coated” tablet) and fitted with 9 mm normal concave punches. To form the lower layer, a blend of 40 parts tizanidine (Farmac Co. Ltd.) powder, 30 parts microcrystalline cellulose (AVICEL™ pH101 FMC International), and 30 parts xylitol (Danisco Sweeteners OY) were granulated with water (5 parts) in a Diosna P1/6 granulator. The granulate was dried in a fluidized bed drier (Aeromatic Laboratory Drier) at 40° C. until the moisture content was less than 1.7 percent. The dry granulate was milled through a 0.8 mm screen. The granulate, 6.6 parts, was mixed with 50 parts compressible sucrose (Nutab™ DMV International), 10 parts microcrystalline cellulose (AVICEL™ pH101 FMC International, 22.4 parts xylitol and 10 parts crospovidone NF (BASF Pharma) and subsequently with one part magnesium stearate. 150 mg of this blend was filled into the die at the first fill station. The placebo layer was formed from a blend of 89 parts compressible sucrose (Nutab™ DMV International), 10 parts polyvinylpyrollidone (POVIDONE K-30, ISP Switzerland AG), and 1 part magnesium stearate. 200 mg of this blend was fed at the second station for a 3 hour delay and about 300 mg was fed at this station to obtain a 6 hour delay. The cylindrical plug was fed as a preformed tablet at the third station using the KILLIAN RUD automatic mechanism for adding preformed tablets to the powder bed. The final tablet was of 9 mm diameter, had a hardness of 10-20 kP, and weighed 400 mg for a 3 hour delay and 500 mg for a six hour delay. The core contained 4 mg tizanidine.

### The Drug Delivery Device

The tablets were coated with an insoluble coating in an Erweka coating pan heated with an air gun. The insoluble coating solution was 3.0 percent ethylcellulose (ETHOCEL™ 7 cps Dow Chemical Inc.) and 0.6 percent polyethylene glycol (PEG 1000 Clariant Hoechst Ltd.) in ethanol. The solution was sprayed through a 1 mm nozzle using 0.5 bar atomizing air with a solution flow of 1-2 ml/minute. The solution flow was varied to prevent sticking of the tablets and to keep the temperature of the tablet bed between 35° to 40° C. The spraying process was stopped at a weight gain of 10-13 mg per tablet, forming the impermeable coat. A drug overcoat was applied over the insoluble coat. The drug overcoat solution was 2.5 weight percent EUDRAGIT® E100 (Rhom Pharma Ltd.) and 1.25 weight percent tizanidine dissolved in ethanol. This solution was sprayed on to the ethylcellulose coated tablets to a weight gain of 12 mg giving a drug content of 4 mg for this layer.

### Results

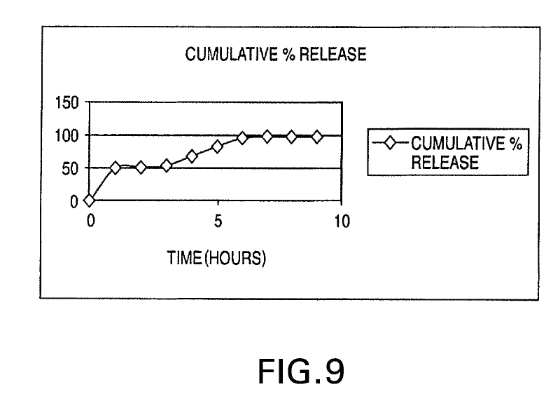

The tablets were tested in a Hanson dissolution bath at 37 C in 900 ml of media. The first two hours were tested in 0.1N HCl. After two hours the tablets were transferred to distilled water. The tizanidine in the samples was determined by an HPLC method on a C-18 column using an aqueous buffer (pH 7.4): methanol system with UV detection at 230 nm. The results are shown in FIG. 9.

The first dose of drug, which is released immediately, is followed by a three hour delay and then a zero order release profile. When the placebo layer is thicker, the delay is longer.

Thus, a drug delivery device is described that gives good control over the time interval between the original burst of drug and a subsequent controlled release of the drug.

### Example 7

### A 36 mg Methylphenidate Hydrochloride Tablet with a Monolayer Plug

The monolayer plug was prepared in a process comprising granulation of methylphenidate hydrochloride and anhydrous lactose with a polyvinylpyrrolidone (PVP, POVIDONE K-30,) solution. The methylphenidate hydrochloride and lactose were mixed for 2 minutes at 380 rpm in a 2 liter Diosna P1/6 mixer vessel. Then, a 5 percent PVP aqueous solution was added gradually over a period of 1 minute, while mixing at the same speed. The wet mixture was then mixed for 30 seconds at 760 rpm. The wet mixture was then dried in a Diosna mini-Lab fluidized bed at 50° C. until L.O.D was lower than 1.5 percent.

The granulate was then milled using a Quadro Comil milling machine through an 813 μm screen at a rate of 3000 rpm. Hydroxypropylcellulose, sodium croscarmellose, methyl cellulose, and microcrystalline cellulose were added to the granulate, and mixed for 5 minutes in a 2 liter V-shaped mixer. Magnesium stearate was added and mixed for an additional 30 seconds. The plug was formed in a KILLIAN RTS 20 tableting machine equipped with FB punches to obtain the following tablet characteristics: Weight—60 mg, Diameter—4.5 mm, and Hardness—3-6 KP.

The formulation of the monolayer plug is presented in Table 17

TABLE 17

| | Distribution | weight |
|---|---|---|
| Ingredient | (%) | (mg) |
| Methylphenidate | 17.50 | 7.00 |
| Lactose anhydrous | 22.10 | 8.84 |
| POVIDONE K-30 | 0.30 | 0.12 |
| AVICEL PH 102 | 5.00 | 2.00 |
| methylcellulose 1500 | 17.10 | 6.84 |
| Ac-di-sol | 19.70 | 7.88 |
| KLUCEL ® HF | 17.55 | 7.02 |
| Magnesium stearate | 0.75 | 0.30 |

The methylphenidate hydrochloride core was prepared in a process comprising granulation of methylphenidate hydrochloride and microcrystalline cellulose, which were granulated in the same manner as the inner plug ingredients, except that purified water was used instead of the PVP solution. The dried granulate was milled by using an Erweka milling machine, equipped with a 0.8 mm screen. The granulate was then mixed with a 75 percent alpha-lactose monohydrate and 25 percent cellulose powder (CELLACTOSE 80™) and compressible sugar and then with magnesium stearate, in the same manner as performed with the plug. The core, with the plug embedded at its surface, was formed in a MANESTY LP 39, a tableting machine that was designed for the production of the methylphenidate hydrochloride tablets. A 7.8 mm Normal-Concave punch was used to obtain the following tablet characteristics: Weight—375 mg, Diameter—7.8 mm, and Hardness—12-19 KP.

The formulation of the core is presented in Table 18

TABLE 18

| | Distribution | Weight |
|---|---|---|
| Ingredient | (%) | (mg) |
| Methylphenidate | 7.30 | 23.00 |
| AVICEL PH 102 | 11.40 | 35.91 |
| Nutab | 40.15 | 126.47 |
| CELLACTOSE 80 ™ | 40.15 | 126.47 |
| Magnesium stearate | 1.00 | 3.15 |
| total | 100.00 | 315.00 |

The core was coated with a water permeable coating consisting of Ethylcellulose and EUDRAGIT® L-100 (1.5 percent each in alcohol 95 percent USP), and triethyl citrate (0.6 percent) as a plasticizer. An Accela-Cota coating system and a Watson 505S peristaltic pump were employed. The coating parameters were: tablet temperature of 30° to 32° C. and a pan speed of 7 rpm. The solution was sprayed through an inner tube diameter 3.1 mm at a solution pump speed of 10-20 rpm, using an atomization pressure of 0.5-1 bar. The tablets were dried for 15 minutes at 30° C. The tablet weight gain at the end of process was 7-11 mg.

The formulation of the water permeable coat is presented in Table 19

TABLE 19

| | (%) | (mg) |
|---|---|---|
| ETHOCEL ™ 7 cps | 41.65 | 3.33 |
| EUDRAGIT ® L-100 | 41.65 | 3.33 |
| triethyl citrate | 16.70 | 1.34 |
| total | 100.00 | 8.00 |

The immediate released overcoat was formed over the water permeable coat, and was consisted of EUDRAGIT® E-100 (2.5 percent in alcohol 95 percent USP) and MPH (1.25 percent). The coating system and parameters for the immediate release coating were the same as for the water permeable coating. Tablet weight gain at the end of process was 16 to 20 mg.

The formulation of the Drug coat is presented in Table 20

TABLE 20

| Ingredient | Distribution | Weight |
|---|---|---|
| Methylphenidate | 33.30 | 6.00 |
| EUDRAGIT ® E-100 | 66.70 | 12.01 |
| total | 100.00 | 18.01 |

Release Method of Methylphenidate Hydrochloride

### Dissolution Parameters

Equipment: 6-vessl assembly, Apparatus 2 (Paddle)
Medium: 2 hours in buffer pH=1.2 (70 ml of fuming 37 percent Hydrochloric acid and 20 g sodium chloride up to 10 L purified water), then additional 10 hours in buffer pH6.8 (9 g sodium hydroxide, 68 g Potassium Phosphate to 10 L purified water).
Volume: 900 ml
Stirring Rate: 100 RPM
Medium Temperature: 37° C.±0.5° C.

### Procedure:

Place one weighed tablet in each vessel containing Buffer pH=1.2 and immediately operate the apparatus for 2 hours then transfer the tablets to corresponding vessel containing Buffer pH6.8 and immediately operate the apparatus for 10 hours.

Unless otherwise specified, 3 ml sample are withdrawn from each vessel and filtered through 20 μm polyethylene cannula dissolution filter.

HPLC parameters for dissolution:

| | |
|---|---|
| Column & Packing: | Hypersil CPS 100 * 4.6 mm, 5 μm |
| Column Temperature: | 19-23° C. |
| Injector Temperature: | 19-23° C. |
| Mobile Phase: | Acetate Buffer:Acetonitrile:Methanol |
| | (50:15:35) |
| Flow Rate: | 1.0 ml/minute |
| Detector: | UV at 210 nm |
| Sample/Injection Volume: | 20 μL |
| Injector Wash Solution: | purified water:methanol (30:70) |
| diluent: | mobile phase |

### HPLC Parameters for IDD:

Mobile Phase is 65:35 Tetra butyl ammonium acetate buffer: Methanol

All the other parameters are identical to the dissolution method

### Dissolution Profile:

| Time | Release |
|---|---|
| (hr) | (%) |
| 0.5 | 21.0 |
| 1.0 | 31.6 |
| 2.0 | 40.4 |
| 3.0 | 50.6 |
| 4.0 | 61.9 |
| 6.0 | 73.1 |
| 8.0 | 77.7 |
| 10.0 | 82.9 |
| 12.0 | 85.2 |
| 18.0 | 84.7 |

### Example 8

### Human PK Trial of Methylphenidate

MATERIALS AND METHODS The tablets described in Example 5 were used in this Pharmacokinetic study.

### Protocol Synopsis

PROTOCOL TITLE: A Single-Dose, Pharmacokinetic study of Methylphenidate HCl (36 mg) in Healthy, Male Volunteers

### Introduction:

CONCERTA® (Alza Pharmaceuticals) is a once-daily tablet formulation of Ritalin® (methylphenidate HCl), for the treatment of attention deficit/hyperactivity disorder (AD/HD) in children. AD/HD, the most commonly diagnosed behavioral disorder in children, with prevalence estimates ranging from 3-7 percent of school-age children, is typically treated pharmacologically, as well as with psychosocial therapies. Among the AD/HD medications prescribed are psychostimulants [such as methylphenidate HCl, dextroamphetamine (Dexedrine®), and amphetamine salts (ADDERALL®)]; tricyclic antidepressants; as well as neuroleptics, tranquilizers and mood stabilizers, as adjunctive medications.

However, methylphenidate HCl is by far the most widely prescribed medication, with reports of about 70-90 percent of AD/HD patients responding positively. Methylphenidate HCl, a mild central nervous system stimulant derived synthetically from amphetamine, and available since the mid-1970's for the treatment of AD/HD, has been shown to decrease impulsivity and hyperactivity, increase attention, and in some children, decrease aggression. Clinical improvement following methylphenidate use has been demonstrated in laboratory cognitive scales, classroom measures of disruption and academic completion, teacher ratings, parent-child interactions, and peer relationships.

Prior to the launch of CONCERTA®, in the fall of 2000, methylphenidate HCl was typically administered as an immediate release tablet of 5, 10, or 20 mg, 2-3 times daily. Immediate release (IR) methylphenidate HCl is absorbed and metabolized quickly (time to peak in children is 1.9 hours, range 0.3-4.4 hours), is excreted fairly rapidly and extensively (in children, 67 percent of the drug; in adults, 80 percent), and is effective from 1 to 4 hours following oral administration, with a pharmacokinetic half-life of 2-3 hours. Due to methylphenidate's relatively short half-life, multiple daily dosing was necessary to ensure adequate therapeutic coverage for the child throughout the school day, including after-school homework hours, and until bedtime.

As a result, there were serious limitations to methylphenidate's use. The need for midday dosing during the school day negatively contributed to poor compliance. In those schools where policy prohibited the administration of psychoactive medication by school personnel, AD/HD children were responsible themselves to take their midday pill, resulting in poor compliance and ineffective treatment. Alternatively, in schools where the health care staff was responsible to administer medication, midday visits to the clinic isolated AD/HD children, stigmatizing them among their peers, as well as imposing upon the school the responsibility of handling a DEA-controlled substance.

The availability, therefore, of a once-a-day dosing formulation of methylphenidate that is clinically effective, by providing a burst release followed by an ascending dosage of methylphenidate up to about 8 hours, that simulates daily dosing of three immediate-release methylphenidate tablets, as is seen in CONCERTA®, clearly offers substantial benefits for AD/HD patients and their health-care providers.

### Study Background

The Teva R&D Initiative Group, Jerusalem, Israel recently developed a generic version of the CONCERTA® tablet, using a proprietary tablet formulation, called “CARP”—Controlled Area Release Plug. In the generic version, 36 mg of methylphenidate HCl is released over 12-18 hours through erosion of the multiple active drug/matrix layers though a defined geometric space. In vitro test results for the R&D Initiative formulation indicate that effectively 20 percent of drug is released immediately from the overcoat within 1-2 hours, followed by 60-70 percent release over the 8-10 hours, with a final 10-20 percent until hours 12-16.

This correlates quite nicely with the release profile exhibited by CONCERTA®, in which ALZA'S OROS® osmotic pressure system is used to deliver methylphenidate HCl at a controlled rate. The in vivo drug release for the current CONCERTA® formulation (36 mg) is essentially a biphasic ascending profile, in which there is an initial maximum concentration at about 1-2 hours, with a gradual increase in levels over the next several hours. Peak plasma concentrations are achieved at about 6-8 hours, followed by a gradual decrease in plasma levels. The overall result is a release of clinically effective plasma levels of methylphenidate over 12-14 hours following initial dosing, with the relative bioavailability of CONCERTA® comparable to three times a day dosing of immediate-release methylphenidate, but with fewer fluctuations between peak and trough concentrations, as compared to IR dosing.

### Study Rationale

The pilot pharmacokinetic study is being conducted to evaluate whether the in vitro release profile observed for the methylphenidate HCl once-daily generic version, can be reproduced in an in vivo system.

The pharmacokinetic data to be evaluated include the Cₘₐₓ, Tₘₐₓ, and AUC (area under the plasma concentration versus time curve) following single dosing of the generic once-daily methylphenidate HCl formulations. The assay will evaluate the levels of the methylphenidate (racemic version) and its main metabolite, ritanilic acid (PPA, piperidine acetic acid).

Previous food effect studies of CONCERTA® indicate that food does not impede drug absorption and that the CONCERTA® may be administered in the fed or fasted state. Thus, the food effect of the once-daily methylphenidate HCl generic version will also be evaluated.

### Study Objective:

The objective of this study is to measure the pharmacokinetics of generic methylphenidate HCl once-daily tablets (36 mg; Teva R&D Initiative), in healthy, adult male volunteers following single dose administration. The pharmacokinetic profiles (Cₘₐₓ, Tₘₐₓ, and AUC) will be evaluated under both fasted and fed conditions.

### Study Design:

Single-center, 2 period, pharmacokinetic study in 12 healthy male volunteers. The first period will be conducted under fasted conditions, while the second period will be conducted under fed conditions.

### Study Duration:

Two treatment periods separated by a minimum 1 week wash-out period between periods. Each treatment period will comprise the following:

I. An in-patient overnight stay from the evening prior to study dosing;

II. Hourly blood sampling for 12 hours following dosing; and

III. Return to clinic following morning for final 24 hour sample.

### Subjects:

Twelve healthy, non-smoking, male volunteers, ages 18-40. Subjects must be in good general health with no concurrent medical conditions. Subjects may not be taking any other concomitant medications during the entire study.

### Dosing Regimen:

All subjects will receive the test 2 treatment periods, with each treatment period, separated by a 1 week wash-out phase. The test article will be administered during the first period under fasting conditions, i.e., first thing in the AM, on an empty stomach, following an overnight fast of at least 10 hours.

The test article will then be administered during the second period, under fed conditions, i.e., 30 minutes after a standard, high-fat breakfast, following an overnight fast of at least 10 hours. In both periods, the treatments will be administered together with 1 glass (240 ml) water.

### Sample Collections and Study Assessments:

At each treatment period, blood for pharmacokinetic analysis will be collected via indwelling intravenous cannula. Whole blood (7 ml) will be collected in labeled vacutainers containing K-EDTA at 0 hour pre-dosing, and then at 15, 30, 60, and 90 minutes, 2, 3, 4, 6, 8, 10, 12, 14, 17, 20, and 24 hours post-dosing (total 16 samples). The blood will be collected at 4° C. to prevent ex vivo methylphenidate degradation. Immediately after collection, samples will be centrifuged at 1500×g for 10 minutes, and the plasma will be removed, divided into two aliquots, and placed separately into polypropylene vials and stored frozen (−20° C.) at the study site. At least 1 set of labeled aliquots will be shipped for analysis from the study site to the analytical laboratory, Anapharm Inc., Quebec, Canada, for assay. The samples should be packaged in sufficient dry ice to ensure that the samples remain frozen for at least 72 hours. The remaining set of aliquots will remain in the freezer at the clinical facility, until further notification from the Sponsor.

The samples collected will be analyzed at Anapharm Inc., using a validated high-performance liquid chromatography tandem mass spectrometry (LC/MS/MS) method in plasma, to determine the concentrations of methylphenidate HCl (racemate) and its main metabolite, ritanilic acid (PPA). The lower limits of detection (LLD) for methylphenidate and its metabolite will be determined by the analytical laboratory.

The chromatographic data will be processed at Anapharm. The audited results of the sample analysis will be provided by Anapharm in a tabular form to the Sponsor. For each session, for each subject, the Cₘₐₓ (maximum concentration) and Tₘₐₓ (time of maximum concentration) will be determined by inspection of the concentration versus time curves. The values obtained for the Cₘₐₓ and Tₘₐₓ for all subjects within a treatment group will be averaged, and the mean Cₘₐₓ and Tₘₐₓ calculated. Similarly, the AUC values for each subject for each session will be assessed and a mean AUC per treatment arm (fed vs. fasted), will be calculated for comparison.

It should be noted that although the study itself will not be blinded, i.e., both the subject and investigator will be aware which treatment the subject is receiving, the blood samples collected will be coded, so that the analyst at Anapharm Inc. performing the assay will be blinded. This will ensure that no bias is introduced in the study analysis.

### Safety Evaluations:

A routine biochemistry, hematology and urinalysis will be conducted at screening (within 21 days of the study) to ensure subject eligibility, and again, at study termination, following the last treatment period, to ensure that there has been no change as a result of the study treatments. Vital signs and a brief physical examination will be conducted at screening and at study termination; additionally, vital signs will be checked prior to each dosing period. The pre-study screening evaluation will also include a one-time HIV screen, hepatitis B, C screen, as well as drugs of abuse screen (to be repeated prior to each study session). All subjects determined to be eligible on the basis of the above noted physical exam and screening laboratory tests, will receive an electrocardiogram prior to the study.

During the study, subjects will be observed by clinic personnel for any adverse reactions that may arise during the treatment sessions. The primary adverse events associated with chronic methylphenidate dosing are nervousness, insomnia, and appetite suppression. During pharmacokinetic studies of the reference article, CONCERTA®, headache, nausea, dizziness and somnolence were the adverse events reported. All adverse events noted will be reported and recorded.

### Study Expectations:

It is expected that the pharmacokinetic profiles (Tₘₐₓ, Cₘₐₓ, and AUC) for once-daily methylphenidate HCl following dosing in either fed or fasted conditions will be similar to literature data for CONCERTA® and that there will be no significant food effect evident.

### Results

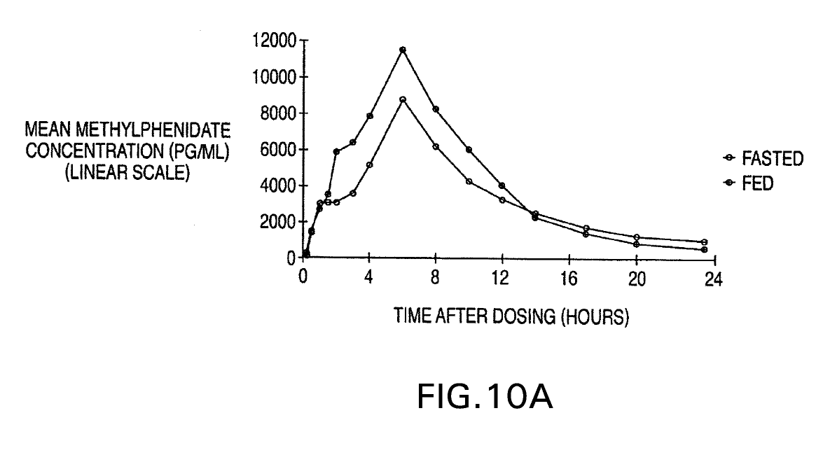

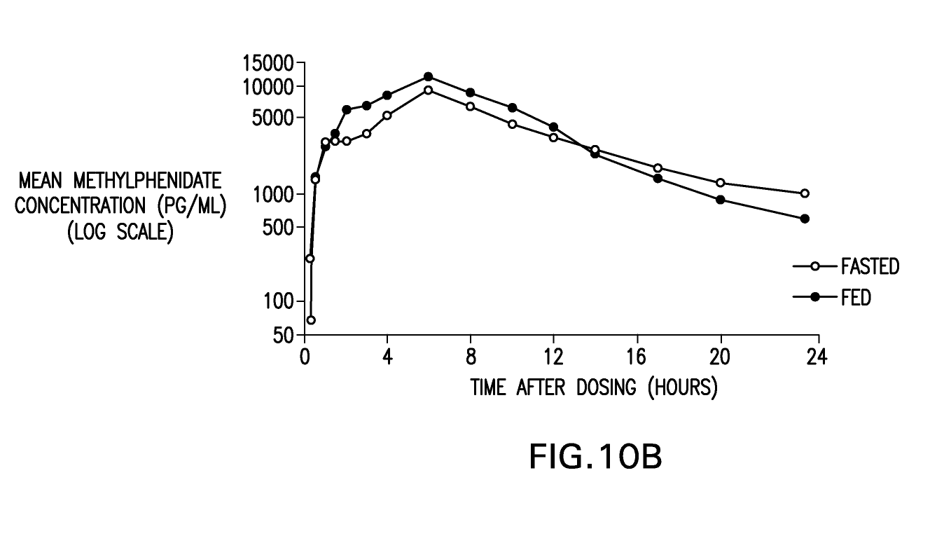

The pharmacokinetic results of the trial are summarized in Table 21. The graphs of the average concentrations of methylphenidate for all volunteers in the fed and fasted state are given in FIGS. 10a and b. A comparison of the results to literature results for CONCERTA® is given in Table 22.

TABLE 21
Pharmacokinetic Parameters for Fed and Fasted
Administration of CARP Methylphenidate

| | AUC | | | | | Tmax | Cmax |
|---|---|---|---|---|---|---|---|
| vol-sess | (h * pg/g) | AUC inf | t½ | CL | Vd | (h) | (pg/g) |
| sub-02-fast | 135768.7 | 146764.0 | 5.5 | 0.3 | 2.1 | 6.0 | 16813.0 |
| sub-04-fast | 71622.3 | 78448.6 | 6.5 | 0.5 | 4.7 | 6.0 | 8894.0 |
| sub-05-fast | 57939.6 | 84075.0 | — | 0.6 | — | 6.0 | 3181.0 |
| sub-06-fast | 89305.1 | 90413.0 | 3.2 | 0.4 | 1.9 | 6.0 | 12980.0 |
| sub-10-fast | 69621.3 | 87148.8 | 8.0 | 0.5 | 6.0 | 6.0 | 5597.0 |
| sub-11-fast | 59955.3 | 66837.1 | 6.4 | 0.6 | 5.5 | 8.0 | 4437.0 |
| sub-01-fast | 76974.8 | 76974.8 | 6.2 | 0.5 | 4.2 | 6.0 | 7638.0 |
| sub-03-fast | 79199.1 | 94600.1 | 8.4 | 0.5 | 5.5 | 6.0 | 9126.0 |
| sub-07-fast | 79263.1 | 82754.1 | 4.6 | 0.5 | 3.0 | 4.0 | 8323.0 |
| sub-08-fast | 43951.9 | 46616.6 | 4.8 | 0.8 | 5.7 | 6.0 | 5429.0 |
| sub-09-fast | 112308.0 | 115488.7 | 3.9 | 0.3 | 1.8 | 6.0 | 16315.0 |
| sub-12-fast | 83772.8 | 89946.2 | 4.2 | 0.4 | 2.6 | 6.0 | 9190.0 |
| sub-01-fed | 84269.0 | 85450.6 | 2.9 | 0.4 | 1.8 | 6.0 | 15547.0 |
| sub-03-fed | 105656.9 | 108586.5 | 3.8 | 0.3 | 1.9 | 6.0 | 15430.0 |
| sub-07-fed | 78667.1 | 82765.7 | 4.0 | 0.5 | 2.7 | 10.0 | 8082.0 |
| sub-08-fed | 73960.2 | 74582.1 | 3.0 | 0.5 | 2.1 | 2.0 | 12273.0 |
| sub-09-fed | 99874.5 | 102764.8 | 3.9 | 0.4 | 2.1 | 8.0 | 10649.0 |
| sub-12-fed | 96907.6 | 110862.0 | 5.7 | 0.4 | 3.1 | 4.0 | 12291.0 |
| sub-02-fed | 168704.9 | 172355.0 | 3.5 | 0.2 | 1.1 | 2.5 | 21000.0 |
| sub-04-fed | 110300.6 | 111340.3 | 2.7 | 0.3 | 1.3 | 6.0 | 21000.0 |
| sub-05-fed | 111532.3 | 114982.0 | 3.4 | 0.3 | 1.6 | 10.0 | 10378.0 |
| sub-06-fed | 103398.1 | 105050.0 | 3.0 | 0.3 | 1.5 | 6.0 | 14265.0 |
| sub-10-fed | 96652.2 | 98841.7 | 3.2 | 0.4 | 1.7 | 6.0 | 12803.0 |
| sub-11-fed | 66807.2 | 69717.3 | 4.7 | 0.5 | 3.7 | 4.0 | 9452.0 |
| AVG(fast) | 79973.5 | 88338.9 | 5.6 | 0.5 | 3.9 | 6.0 | 8993.6 |
| AVG (fed) | 99727.6 | 103108.2 | 3.7 | 0.4 | 2.0 | 5.9 | 13597.5 |
| geomn(fast) | 76778.9 | 85317.8 | 5.4 | 0.5 | 3.6 | 5.9 | 8034.9 |
| geomn(fed) | 97010.5 | 100345.7 | 3.6 | 0.4 | 1.9 | 5.3 | 13059.3 |
| stddev(fast) | 24552 | 24638 | 1.7 | 0.1 | 1.7 | 0.9 | 4388 |
| stddev(fed) | 26121 | 26578 | 0.9 | 0.1 | 0.8 | 2.6 | 4134 |

TABLE 22
Comparison of Parmacokinetic Parameters of Methylphenidate CARP
to Literature Parameters for CONCERTA ®

| | | | | Concetrta ® | | |
|---|---|---|---|---|---|---|
| Fasted/ | | CARP (36 mg) | | (Literature*) | | |
| fed | Parameter | mean | SD | mean | SD | ratio |
| fasted | Cmax (ng/ml) | 9.00 | 4.39 | 6.20 | 2.20 | 145.2% |
| fed | Cmax (ng/ml) | 13.60 | 4.13 | 6.87 | 2.30 | 198.0% |
| | ratio | 151.1% | | 110.8% | | |
| fasted | AUCI (ng · h/ml) | 88.34 | 24.63 | 67.6 | 23.70 | 130.7% |
| fed | AUCI (ng · h/ml) | 103.11 | 26.58 | 79.0 | 26.80 | 130.5% |
| | ratio | 116.72% | | 116.9% | | |

*Modi NB et. al. Effect of food on the pharmacokinetics of osmotic controlled release methylphenidate HCl in healthy subjects. Biopharmaceutics and Drug Disposition, (2000), 21, 23-31

The results of this trial show methylphenidate release into the plasma at amounts and rates similar to that of CONCERTA®. The total amount of methylphenidate found in each volunteer as expressed by the area under the concentration time curve extrapolated to infinity (AUC_{inf}) ranged from ranged from 47 to 147 h*ng/g for the fasted subjects and from 70 to 172 h*ng/g for the fed subjects, with an average value of 88 and 103 h*ng/g respectively. The maximum concentrations found in the plasma ranged from 3.2 ng/g to 16.8 ng/g for the fasted subjects and 8.1 to 21.0 ng/g for the fed subjects, with respective averages of 9.0 and 13.6 ng/g. The time of maximum concentration ranged from 4 to 8 hours (with 10 of twelve subjects having Tₘₐₓ at 6 hours) in the fasted subjects and from 2 to 10 hours in the fed subjects. The average Tₘₐₓ was 6.0 for the fasted subjects and 5.9 for the fed subjects. The half life of elimination ranged from 3.2 to 8.4 hours in the fasted subjects and 2.7 to 5.7 hours in the fed subjects with averages of 5.6 and 3.7 hours respectively. Comparisons of these values to those of literature values of CONCERTA® did not show bioequivalence but the results are close and encouraging for a pilot trial.

### CONCLUSIONS

The results show a successful in vivo profile of controlled release of methylphenidate. As designed, and seen in vitro, the in vivo profile shown in FIGS. 10 (a) and (b) is one of an initial burst of drug followed, after a delay, by an extended release that gives an ascending profile in the plasma. This confirms the usefulness of this drug delivery device for providing complicated drug delivery profiles.

### Example 9

### Human PK Trial of 36 mg Methylphenidate Hydrochloride Tablets with Monolayer Plug

MATERIALS AND METHODS The tablets described in Example 7 were used in this Pharmacokinetic study.

### Protocol Synopsis

### Protocol Title:

A Single-Dose, Three-Way Crossover Comparative Bioavailability Study of Two Novel Test Formulations of Methylphenidate HCl (36 mg; Teva R&D Initiative) vs. CONCERTA (Methylphenidate HC136 mg, Alza Pharmaceuticals, Lot number 06L7192) in Healthy, Male Volunteers.

### Study Objective:

The objective of this study is to measure the pharmacokinetics of generic methylphenidate HCl once-daily tablets (36 mg; Teva R&D Initiative), in healthy, adult male volunteers following single dose administration. The pharmacokinetic profiles (Cₘₐₓ, Tₘₐₓ, and AUC) will be evaluated under both fasted and fed conditions.

### Study Design:

Pharmacokinetic study in 12 healthy male volunteers under fed and fasted conditions.

### Results

The pharmacokinetic results of the trial are summarized in Table 23.

TABLE 23

| | | Data (TEST) | | |
|---|---|---|---|---|
| | | | 36 mg unilayer | Data (REF) |
| | | 36 mg bilayer | (monolayer) | CONCERTA |
| Fasted/fed | parameter | inner (plug) | inner | (Lot Number 6L7192) |
| fasted | Cₘₐₓ (ng/ml) | 8.92 | 8.44 | 8.10 |
| | AUC₀₋ₜ (ng · h/ml) | 86.56 | 84.65 | 96.64 |
| | AUC_{0-inf} (ng · h/ml) | 88.49 | 86.16 | 98.14 |
| | Tₘₐₓ (hours) | 6.21 | 6.0 | 7.83 |
| | Cₘₐₓ FAST ratio (% test/ref) | 109.97 | 103.93 | |
| | 90% CI | 96.98-124.74 | 91.65-117.86 | |
| | AUC₀₋ₜ FAST ratio | 90.00 | 87.48 | |
| | (% test/ref) | | | |
| | 90% CI | 85.02-95.27 | 82.63-92.60 | |
| | AUC_{0-tinf} FAST ratio | 90.56 | 87.72 | |
| | (% test/ref) | | | |
| | 90% CI | 85.58-95.83 | 82.90-92.82 | |
| | AUC pr FAST ratio | 113.4 | 112.62 | |
| | (% test/ref) | | | |
| | 90% CI | 102.17-125.86 | 101.47-124.99 | |
| fed | Cₘₐₓ (ng/ml) | 9.54 | 8.57 | 8.21 |
| | AUC₀₋ₜ (ng · h/ml) | 86.48 | 95.16 | 96.43 |
| | AUC_{0-inf} (ng · h/ml) | 89.92 | 96.49 | 98.04 |
| | Tₘₐₓ (hours) | 5.38 | 5.04 | 7.71 |
| | Cₘₐₓ FED ratio (% test/ref) | 111.92 | 102.78 | |
| | 90% CI | 94.98-131.89 | 87.22-121.12 | |
| | AUC₀₋ₜ FED ratio | 90.02 | 99.23 | |
| | (% test/ref) | | | |
| | 90% CI | 85.88-94.36 | 94.67-104.01 | |
| | AUC_{0-tinf} FED ratio | 91.91 | 98.95 | |
| | (% test/ref) | | | |
| | 90% CI | 86.84-97.28 | 93.49-104.72 | |
| | AUC pr FED ratio (% test/ref) | 106.69 | 112.31 | |
| | 90% CI | 93.91-121.22 | 98.85-127.60 | |

The total amount of methylphenidate found in each volunteer as expressed by the area under the concentration time curve extrapolated to infinity (AUC_{inf}) had an average value of 88.49 ng*h/ml and 86.16 ng*h/ml for the fasted subjects of the bilayer and monolayer inner tablets, respectively. The value for CONCERTA® in the fasted state was 98.14 ng*h/ml. The average values of AUC_{inf} for the fed subjects were 89.92 ng*h/ml and 96.49 ng*h/ml for the bilayer and monolayer inner tablets, respectively. The value for CONCERTA® in the fed state was 98.04 ng*h/ml. The maximum concentrations found in the plasma (Cₘₐₓ) of fasted subject for the bilayer and monolayer inner tablets had respective averages of 8.92 and 8.44 ng/ml. Cₘₐₓ value for CONCERTA® in the fasted state was 8.10. The fed study resulted in average Cₘₐₓ values for the bilayer and monolayer inner tablets of 9.54 and 8.57 ng/ml, respectively. The fed study of CONCERTA® resulted in a Cₘₐₓ value of 8.21 ng/ml. The time of maximum concentration (Tₘₐₓ) for the fasted subjects had respective averages of 6.21 and 6.0 hours for the bilayer and monolayer inner tablets. The fasted study of CONCERTA® resulted in a Tₘₐₓ value of 7.83 hours. The fed study resulted in respective Tₘₐₓ averages of 5.38 and 5.04 hours for the bilayer and monolayer inner tablets. The fed study of CONCERTA® resulted in a Tₘₐₓ value of 7.71 hours.

### CONCLUSION

For each study, the ratios of AUC_{(test/ref)} and C_{max (test/ref)} for both methylphenidate tablets comprising bilayer and monolayer inner tablets were within the permitted range for bioequivalence with CONCERTA®.

### Example 10

### Large Scale Production of 36 mg Methylphenidate Hydrochloride Tablet with a Monolayer Plug

The formulation and analytical methods are according to those of example 7.

The monolayer plug was prepared in a process comprising granulation of methylphenidate hydrochloride and anhydrous lactose with a polyvinylpyrrolidone (PVP, POVIDONE K-30) solution. The methylphenidate hydrochloride and lactose were mixed for 3 minutes at 260 rpm in a 6 liter Diosna P1/6 mixer vessel. Then, a 5 percent PVP aqueous solution was added gradually over a period of 1 minute, while mixing at the same speed. The wet mixture was then mixed for 15 seconds at the same speed. The wet mixture was then dried in a Diosna mini-Lab (23 L bowl) fluidized bed at 50° C. until L.O.D was lower than 1.0 percent. The granulate was then milled using a Quadro U20 milling machine through an 813 μm screen at a rate of 1000 to 3000 rpm. The granulate was introduced into a 20 L Flow Bin and mixed with Hydroxypropylcellulose, sodium croscarmellose, methyl cellulose, and microcrystalline cellulose for 15 minutes in a Bin Blender at a speed of 10 rpm. Magnesium stearate was sieved through a 50 mesh sieve screen and then was transferred to the Flow Bin and mixed for an additional 3 minutes at the same speed. The plug was formed in a KILLIAN RTS 20 tableting machine equipped with 4.5 mmFB punches to obtain the following tablet characteristics: Weight—40 mg, Diameter—4.5 mm, and Hardness—2-4 SCU.

The methylphenidate hydrochloride core was prepared in a process comprising granulation of methylphenidate hydrochloride and microcrystalline cellulose, which were granulated in the same manner as the inner plug ingredients, except that purified water was used instead of the PVP solution, and that the mixer employed was a Diosna P100 at speed I for dry mixing and purified water addition and at speed II for further 90 sec of massing following water addition. The granulate was then dried in a GPCG PRO-30 Fluidized Bed at 50° C. until L.O.D was lower than 1.0 percent. The dried granulate was milled by using a FREWITT milling machine, equipped with a 0.8 mm screen. The granulate was then mixed in a 100 L Flow Bin with a 75 percent alpha-lactose monohydrate and 25 percent cellulose powder (CELLACTOSE 80) and compressible sugar for 15 minutes in a Bin Blender at 10 rpm. The magnesium stearate was sieved and then mixed with the other ingredients for additional 3 minutes at the same speed. The core, with the plug embedded at its surface, was formed in a MANESTY LP 39, a tableting machine that was designed for the production of the methylphenidate hydrochloride tablets. A 7.8 mm Normal-Concave punch was used to obtain the following tablet characteristics: Weight—355 mg, Diameter—7.8 mm, and Hardness—17-22 SCU

The core was coated with a water permeable coating consisting of Ethylcellulose and EUDRAGIT® L-100 (1.5 percent each in alcohol 95 percent USP), and triethyl citrate (0.6 percent) as a plasticizer. An O′HARA LAB COAT 32 BIN coating system outfitted with 2×1.2 mm nozzles and a Watson 505S peristaltic pump were employed. The coating parameters were: inlet air temperature of 50° C., outlet air temperature of 28-36° C. and a pan speed 2-4 rpm. The solution was sprayed at a rate of 40-90 g/minute, using an atomization pressure of 1-2 bar. The tablets were dried at minimum drum speed and temperature until an outlet temperature of 25-30 is reached. The tablet weight gain at the end of process was 7-11 mg. The tablets were coated in two sublots, each sublot comprising about 75,000 tablets.

The immediate released drug coat was formed over the water permeable coat, and was consisted of EUDRAGIT® E-100 (2.5 percent in alcohol 95 percent USP) and MPH (1.25 percent). An O′HARA Coating Machine 48″ coating system outfitted with 4×1.0 mm nozzles and a Watson 505S peristaltic pump were employed. The coating parameters were the same as for the water permeable coating, only that the spray rate during the immediate released drug coat process was 100-300 g/minute. Tablet weight gain at the end of process was 16 to 20 mg.

### Dissolution Profile:

| Time | Release |
|---|---|
| (hr) | (%) |
| 0.5 | 32.9 |
| 1.0 | 38.1 |
| 2.0 | 45.3 |
| 3.0 | 49.4 |
| 4.0 | 53.0 |
| 6.0 | 59.9 |
| 8.0 | 68.3 |
| 10.0 | 75.1 |
| 12.0 | 79.1 |

Total impurities at production (t=0) were 0.3 percent by weight of methylphenidate content of the final tablet.

Total impurities at 6 days in 70° C. (in oven) were 1.3 percent by weight of methylphenidate content of the final tablet.

### Example 11

### A 36 mg Methylphenidate Hydrochloride Tablet with a Monolayer Plug and an Aqueous Drug Coating

The monolayer plug was prepared in a process comprising compaction of lactose anhydrous, POVIDONE, microcrystalline cellulose, methyl cellulose, sodium croscarmellose, Hydroxypropylcellulose, methylphenidate hydrochloride, butylated hydroxytoluene, and half of the amount of magnesium stearate. The compaction was performed by using a Roller Compactor (WP 200 Pharma, Alexanderwerk) outfitted with a grooved and knurled surface and corrugated pressing rolls. The slug layer thickness was 4 mm. The slug was then milled using a QUADRO U10 milling machine through a 6350 μm screen at a rate of 2000 rpm and then through a 1397 μm screen at the same speed. The milled slug was mixed with the remaining magnesium stearate in a 50 L Bin Mixer for 3 minutes. The plug was formed in a KILLIAN PARISSIMA tableting machine equipped with 4.5 FB punches to obtain the following tablet characteristics: Weight—40 mg, Diameter—4.5 mm, and Hardness—2-5 SCU.

The formulation of the monolayer plug is presented in Table 24

TABLE 24

| Ingredient | distribution (%) | Weight (mg) |
|---|---|---|
| Methylphenidate | 17.50 | 7.00 |
| Lactose anhydrous | 22.10 | 8.84 |
| POVIDONE PVP | 0.30 | 0.12 |
| K-30 | | |
| AVICEL PH 102 | 5.00 | 2.00 |
| Methylcellulose | 17.10 | 6.84 |
| 1500 | | |
| AC-DI-SOL | 19.70 | 7.88 |
| KLUCEL ® HF | 17.50 | 7.00 |
| BHT | 0.10 | 0.04 |
| Magnesium stearate | 0.70 | 0.28 |

The methylphenidate hydrochloride core was prepared in a process comprising compaction of methylphenidate hydrochloride, microcrystalline cellulose, and two thirds of the amount of magnesium stearate. The compaction was performed by using a Roller Compactor (WP 200 Pharma, Alexanderwerk) outfitted with a grooved and knurled surface and corrugated pressing rolls. The slug layer thickness was 2.6 mm. The slug was then milled using a Quadro U10 milling machine through a 6350 μm screen at a rate of 2000 rpm and then through a 1397 μm screen at the same speed. Then, the compacted mixture was mixed in a 50 L Bin Mixer with a 75 percent alpha-lactose monohydrate and 25 percent cellulose powder (a 75 percent alpha-lactose monohydrate and 25 percent cellulose powder 80), compressible sugar, butylated hydroxytoluene for 20 min. The remaining magnesium stearate was then added and mixed for another 3 minutes. The core, with the plug embedded at its surface, was formed in a MANESTY LP 39, a tableting machine that was designed for the production of the methylphenidate hydrochloride tablets. A 7.8 mm Normal-Concave punch was used to obtain the following tablet characteristics: Weight—355 mg, Diameter—7.8 mm, and Hardness—16-18 SCU.

The formulation of the core is presented in Table 25

TABLE 25

| Ingredient | distribution (%) | Weight (mg) |
|---|---|---|
| Methylphenidate | 6.83 | 21.51 |
| AVICEL PH 102 | 10.67 | 33.61 |
| Magnesium stearate | 0.47 | 1.48 |
| Di-pac | 40.15 | 126.47 |
| CELLACTOSE | 41.38 | 130.35 |
| 80 ™ | | |
| BHT | 0.04 | 0.13 |
| Magnesium stearate | 0.46 | 1.45 |

The core was coated with a water permeable coating consisting of Ethylcellulose and EUDRAGIT® L-100 (4.5 percent each in alcohol 95 percent USP), and triethyl citrate (1.8 percent) as a plasticizer. An Accela-Cota coating system and a Watson 505S peristaltic pump were employed. The coating parameters were: tablet temperature of 28° to 32° C. and a pan speed of 6-12 rpm. The solution was sprayed through an inner tube diameter 3.1 mm at a solution pump speed of 40-70 rpm, using an atomization pressure of 0.5-1 bar. The tablets were dried for 5 minutes at 28-32° C. The tablet weight gain at the end of process was 9 mg.

The formulation of the water permeable coat is presented in Table 26

TABLE 26

| Ingredient | distribution (%) | Weight (mg) |
|---|---|---|
| ETHOCEL ™ | 41.67 | 3.33 |
| Premium 7CPS | | |
| EUDRAGIT ® L-100 | 41.67 | 3.33 |
| Triethyl citrate | 16.66 | 1.33 |

The immediate released overcoat was formed over the water permeable coat, and was consisted of Hydroxypropylmethyl cellulose of low-viscosity, such as METHOCEL™ E-5 (1.5 percent in purified water), polyethylene glycol 6000 (0.5 percent) and MPH (12.3 percent). The coating system and parameters for the immediate release coating were the same as for the water permeable coating. Tablet weight gain at the end of process was 8.7 mg.

The formulation of the aqueous drug coat is presented in Table 27

TABLE 27

| Ingredient | distribution (%) | Weight (mg) |
|---|---|---|
| Methylphenidate | 86.00 | 7.48 |
| METHOCEL ™ E-5 | 10.50 | 0.89 |
| PEG 6000 | 3.50 | 0.30 |

Release Method of Methylphenidate Hydrochloride
Equipment: 6-vessl assembly, Apparatus 2(Paddle)
Medium: Medium: 2 hours in buffer pH=1.2 (70 ml of fuming 37 percent Hydrochloric acid and 20 g sodium chloride up to 10 L purified water), then additional 10 hours in buffer pH6.8 (9 g sodium hydroxide, 68 g Potassium Phosphate to 10 L purified water).
Volume: 900 ml
Stirring Rate: 100 RPM
Medium Temperature: 37° C.±0.5° C.

### Procedure:

Place one weighed tablet in each vessel containing Buffer pH=1.2 and immediately operate the apparatus for 2 hours then transfer the tablets to corresponding vessel containing Buffer pH6.8 and immediately operate the apparatus for 10 hours. Unless otherwise specified, 3 ml sample are withdrawn from each vessel and filtered through 20 μm polyethylene (PE) cannula dissolution filter.

### HPLC Parameters for Dissolution:

| | |
|---|---|
| Column & Packing: | Luna CN, 250 * 4.6 mm, 5 μm |
| Column Temperature: | 19-23° C. |
| Temperature: | 19-23° C. |
| Mobile Phase: | Acetate Buffer:Acetonitrile:Methanol |
| | (30:30:40) |
| Flow Rate: | 1.0 ml/minute. |
| Detector: | UV at 210 nm, 10 mm flow cell path length |
| Sample/Injection Volume: | 20 μL |
| Injector Wash Solution: | purified water:methanol (30:70) |
| diluent: | mobile phase |

### HPLC Parameters for IDD:

| | |
|---|---|
| Column & Packing: | YMC Pack-ODS-A 250 * 4.6 mm, 5 μm |
| Column Temperature: | 40° C. |
| Injector Temperature: | 5° C. |
| Mobile Phase: | Potassium Phosphate Buffer |
| | pH 2.0:Methanol 850:250 |
| Flow Rate: | 1.0 ml/minute. |
| Detector: | UV at 209 nm |
| Sample/Injection Volume: | 20 μL |
| Injector Wash Solution: | purified water:methanol (30:70) |
| diluent: | mobile phase |

### Dissolution Profile:

| Time | Release |
|---|---|
| (hr) | (%) |
| 0.5 | 21.8 |
| 1.0 | 32.6 |
| 2.0 | 39.4 |
| 3.0 | 42.7 |
| 4.0 | 49.3 |
| 6.0 | 61.4 |
| 8.0 | 77.7 |
| 10.0 | 84.7 |
| 12.0 | 89.5 |

Total impurities at production (t=0) were 0.11 percent by weight of methylphenidate content of the final tablet.

Total impurities after one month at 40° C. and 75 percent relative humidity were 0.14 percent by weight of methylphenidate content of the final tablet.

We claim:

1. A methylphenidate controlled release drug delivery device, comprising:
a core, a plug, a coating layer, and an outer drug layer, wherein:
a) the core, the plug and the outer drug layer comprise methylphenidate or a pharmaceutically acceptable salt thereof;
b) the plug is embedded in the core;
c) the coating layer is a delay release layer, essentially impermeable to the drug and at least partially surrounding both the core and the plug;
d) the outer drug layer covers at least a portion of the coating; and
e) the plug, when coated, punctures the coating upon swelling of the plug, thereby forming an orifice in the coating.

2. The methylphenidate drug delivery device according to claim 1, wherein the plug is in the form of a swellable, monolayer plug.

3. The methylphenidate drug delivery device according to claim 1, wherein the plug is in the form of a cylinder.

4. The methylphenidate drug delivery device according to claim 1, wherein the core is in the form of a monolayer core.

5. The methylphenidate drug delivery device according to claim 1, wherein the coating layer has a pH dependant water permeability.

6. The methylphenidate drug delivery device according to claim 1, wherein the coating layer comprises an enteric coating ingredient.

7. The methylphenidate drug delivery device according to claim 6, wherein the enteric coating ingredient is a polymer.

8. The methylphenidate drug delivery device according to claim 1, wherein the coating layer comprises an anionic copolymer based on methacrylic acid and methyl methacrylate, such as poly(methacrylic acid, methyl methacrylate) 1:1.

9. The methylphenidate drug delivery device according to claim 6, wherein the enteric coating ingredient is in amount of about 30 to about 70 weight percent based on the weight of the coating layer.

10. The methylphenidate drug delivery device according to claim 1, wherein the coating layer comprises at least one plasticizer.

11. The methylphenidate drug delivery device according to claim 10, wherein, when present, the plasticizer comprises triethylcitrate.

12. The methylphenidate drug delivery device according to claim 1, wherein the coating layer comprises ethylcellulose, an anionic methacrylate copolymer, and a plasticizer.

13. The methylphenidate drug delivery device according to claim 1, wherein the outer drug layer is applied to the coating from a solution comprising at least one coating ingredient and a solvent.

14. The methylphenidate drug delivery device according to claim 13, wherein the solvent comprises water or a mixture of water and ethanol.

15. The methylphenidate drug delivery device according to claim 14, wherein the water is purified water.

16. The methylphenidate drug delivery device according to claim 13, wherein the solvent is selected from the group consisting of a mixture of water and ethanol in ratio of about 95 weight percent to about 5 weight percent, about 80 weight percent to about 20 weight percent, about 60 weight percent to about 40 weight percent and about 50 weight percent to about 50 weight percent and 100 weight percent water.

17. The methylphenidate drug delivery device according to claim 13, wherein the solvent contains ethanol in an amount of 50 weight percent or less.

18. The methylphenidate drug delivery device according to claim 13, wherein the outer drug layer is sprayed onto the coating.

19. The methylphenidate drug delivery device according to claim 1, wherein the outer drug layer comprises at least one of a coating polymer and a plasticizer.

20. The methylphenidate drug delivery device according to claim 19, wherein the coating polymer selected from the group consisting of hydroxyproylmethylcellulose, hydroxy propyl cellulose.

21. The methylphenidate drug delivery device according to claim 19, wherein when present, the coating polymer comprises hydroxypropylmethyl cellulose, and the plasticizer comprises polyethylene glycol.

22. The methylphenidate drug delivery device according to claim 19, wherein when present, the coating polymer is in amount of about 3 to about 60 weight percent based on the total amount of the outer drug layer.

23. The methylphenidate drug delivery device according to claim 19, wherein when present, the plasticizer is in amount of about 2 to about 10 weight percent based on the total amount of the outer drug layer.

24. The methylphenidate drug delivery device according to claim 13, wherein the total impurities after one month at 40° C. and 75 percent relative humidity are less than about 0.5 percent by weight of methylphenidate content of the final tablet.

25. The methylphenidate drug delivery device according to claim 13, wherein the total impurities do not increase by more than about 100 percent by weight of methylphenidate content of the final tablet after one month at 40° C. and 75 percent relative humidity.

26. The methylphenidate drug delivery device according to claim 13, wherein the amount of total impurities at production (t=0) is less than about 0.25 percent by weight of the methylphenidate content of the final tablet.

27. The methylphenidate drug delivery device according to claim 1, wherein the plug comprises at least one of a filler, a binder, a disintegrating agent, a hydrogel forming agent, an anti-oxidant, and a lubricant.

28. The methylphenidate drug delivery device according to claim 27, wherein, when present, the filler comprises anhydrous lactose, the binder comprises polyvinylpyrrolidone, the disintegrating agent comprises at least one of microcrystalline cellulose, methyl cellulose, and sodium croscarmellose, the hydrogel forming agent comprises hydroxypropylcellulose, the anti-oxidant comprises butylated hydroxytoluene, and the lubricant comprises magnesium stearate.

29. The methylphenidate drug delivery device according to claim 1, wherein the core further comprises at least one of a binder, a filler, an anti-oxidant, and a lubricant.

30. The methylphenidate drug delivery device according to claim 29, wherein, when present, the binder comprises microcrystalline cellulose, the filler comprises at least one of compressible sugar and a mixture of alpha-lactose monohydrate and cellulose powder, the anti-oxidant comprises butylated hydroxytoluene, and the lubricant comprises magnesium stearate.

31. The methylphenidate drug delivery device according to claim 1, wherein the plug comprises about 0.1 to about 35 weight percent of methylphenidate hydrochloride, the core comprises 1 to about 15 percent methylphenidate hydrochloride; and the outer drug layer comprises about 0.5 to about 30 weight percent methylphenidate hydrochloride of the total weight of the drug delivery device.

32. The methylphenidate drug delivery device according to claim 1, wherein the plug comprises about 1 to about 5 weight percent of methylphenidate hydrochloride, the core comprises about 1 to about 10 percent methylphenidate hydrochloride; and the outer drug layer comprises about 0.5 to about 5 weight percent methylphenidate hydrochloride of the total weight of the drug delivery device.

33. The methylphenidate drug delivery device according to claim 1, wherein the plug diameter of about 2 to about 8 mm, a plug length or height of about 1.8 to about 5 mm, an outer diameter of about 5 to about 11 mm, an overall length or height of about 4.5 to about 8.5 min, weight gain water permeable coating of about 3 to about 17 mg, and weight gain immediate released over coat of about 3 to about 60 mg.

34. The methylphenidate drug delivery device according to claim 1, comprising about 18 mg, about 27 mg, about 36 mg and about 54 mg of methylphenidate hydrochloride.

35. The methylphenidate drug delivery device according to claim 1, wherein at least one of the core, plug, coating layer, and outer drug layer comprising an antioxidant.

36. The methylphenidate drug delivery device according to claim 1, wherein about 35 percent or less of methylphenidate is released from the drug delivery device over the first 30 min, between about 25 and about 40 percent is released after 1 hour, between about 35 and about 50 percent is released after 2 hours, between about 40 and about 65 percent is released after 4 hours, and 65 percent or above is released after 8 hours when tested in Apparatus 2 (Paddle) at 37° C., 100 RPM in 900 ml of media, when the first two hours are tested in buffer pH=1.2 (70 ml of fuming 37 percent Hydrochloric acid and 20 g sodium chloride in 10 L purified water), then tested for an additional 10 hours in buffer pH 6.8 (9 g sodium hydroxide, 68 g Potassium Phosphate to 10 L purified water).

37. The methylphenidate drug delivery device according to claim 1, wherein the plug comprises about 2 to about 12 mg of methylphenidate hydrochloride, the core comprises about 8 to about 35 mg methylphenidate hydrochloride; and the outer drug layer comprises about 2 to about 14 mg methylphenidate hydrochloride.

38. The methylphenidate drug delivery device according to claim 1, wherein the plug comprises about 2.5 to about 4.5 mg of methylphenidate hydrochloride, the core comprises about 9.8 to about 11.8 mg methylphenidate hydrochloride; and the drug layer comprises about 2.7 to about 4.7 mg methylphenidate hydrochloride.

39. The methylphenidate drug delivery device according to claim 1, wherein the plug comprises about 4 to about 6 mg of methylphenidate hydrochloride, the core comprises about 15 to about 17 mg methylphenidate hydrochloride; and the drug coating layer comprises about 4 to about 6 mg methylphenidate hydrochloride.

40. The methylphenidate drug delivery device according to claim 1, wherein the plug comprises about 6 to about 8 mg of methylphenidate hydrochloride, the core comprises about 20.5 to about 22.5 mg methylphenidate hydrochloride; and the drug coating layer comprises about 6.5 to about 8.5 mg methylphenidate hydrochloride.

41. The methylphenidate drug delivery device according to claim 1, wherein the plug comprises about 9 to about 11 mg of methylphenidate hydrochloride, the core comprises about 31 to about 33 mg methylphenidate hydrochloride; and the drug coating layer comprises about 10 to about 12 mg methylphenidate hydrochloride.

42. A process for applying a drug layer comprising methylphenidate or a pharmaceutically acceptable salt thereof to a drug delivery device comprising applying the drug layer from a solution comprising methylphenidate or a pharmaceutically acceptable salt thereof, at least one coating ingredient and a solvent selected from the group consisting of water or a mixture of water and ethanol.

43. The process according to claim 42, wherein the solvent contains ethanol in an amount of 50 weight percent or less.

44. The process according to claims 42, wherein the solvent is purified water.

45. A method of treating conditions mediated by methylphenidate by administering to a patient in need of treatment the methylphenidate drug delivery device of claim 1.

46. The methylphenidate drug delivery device according to claim 1 for use in treating conditions mediated by methylphenidate selected from the group consisting of attention-deficit hyperactivity disorder, Postural Orthostatic Tachycardia Syndrome, narcolepsy, lethargy, depression, neural insult, and obesity.
